(19) United States
(12) Patent Application Publication
(10) Pub. No.: US 2023/0274842 A1
(43) Pub. Date: Aug. 31, 2023

(54) TISSUE-SCALE, PATIENT-SPECIFIC MODELING AND SIMULATION OF PROSTATE CANCER GROWTH

(71) Applicants: Board of Regents, The University of Texas System, Austin, TX (US); UNIVERSITY OF A CORUNA, A Coruna (ES); Purdue Research Foundation, West Lafayette, IN (US); University of Pavia, Pavia (IT)

(72) Inventors: Thomas J.R. Hughes, Austin, TX (US); Hector Gomez, West Lafayette, IN (US); Guillermo Lorenzo, Austin, TX (US); Alessandro Reali, Pavia (IT); Thomas E. Yankeelov, Austin, TX (US)

(21) Appl. No.: 18/135,580

(22) Filed: Apr. 17, 2023

Related U.S. Application Data
Application No. PCT/ES16/70609 is a continuation-in-part of application No. 16/327,875, filed on Feb. 25, 2019, now Pat. No. 11,631,502.

(51) Int. Cl. G16H 50/50; G16H 50/30

(52) U.S. Cl. CPC G16H 50/50; G16H 50/30

(57) ABSTRACT

Techniques for simulation of the evolution of a tumor in a prostate gland of a subject are disclosed. The techniques may be based on implementation of a coupled system of reaction-diffusion equations and a patient-specific geometric model of the prostate gland of the subject. The use of reaction-diffusion equations and a patient-specific geometric model provides a tumor model that predicts the expected progression of prostate cancer in the subject. The tumor model may be used to devise a customized treatment for the subject.

### RELATED PATENTS

This patent application is a continuation-in-part of U.S. patent application Ser. No. 16/327,875 filed Feb. 25, 2019, which is a national stage filing under 35 USC 371 of International Patent Application No. PCT/ES2016/070609 filed Aug. 24, 2016, both of which are incorporated by reference as if fully set forth herein.

### BACKGROUND

### 1. Field of the Invention

The invention generally relates to mathematical modeling and simulation of diseases and their treatments. More particularly, the invention relates to mathematical modeling and simulation of prostate cancer.

### 2. Description of the Relevant Art

According to the World Health Organization (WHO), prostate cancer (PCa) is the second most common cancer among men worldwide. In 2012, there was an estimate of 1,095,000 new cases and 308,000 deaths worldwide associated with this cancer. In 2016, PCa will be responsible for about 180,890 new cases and 26,120 deaths in the U.S.A.

In most cases, PCa is an adenocarcinoma, a form of cancer that originates and develops in epithelial tissues with glandular organization, for example, the prostatic tissue in charge of producing certain substances in semen. Adenocarcinomas arise as a result of genetic alterations in cell nuclei, such as mutations and epigenetic changes. Additionally, the influence of several environmental factors, such as diet, may promote further modifications of the genome. Tumoral cells exhibit an aberrant and competitive behavior, characterized by overproliferation and an invasive demeanor. Consequently, they may endanger the structure and the normal operation of the tissue they belong to, eventually jeopardizing the survival of the whole organism. The same tumor may present different groups of cancerous cells, with different metabolism, local environment and aggressiveness. Malignant cells may evolve during their life cycle, and thus belong to a new group. The structure, behavior and evolution of a tumor depend on the specific genetic alterations that may have originated it and supported its evolution.

The development of a prostate adenocarcinoma requires a gradual accumulation of mutations in a number of different genes, which varies from patient to patient, but is usually at least seven. As a result of these alterations of the genome over years, an initial moderate disorder of cell behavior evolves gradually towards an advanced cancer. As the tumor develops, it becomes more malignant and cell differentiation decreases. This evolutive phenomenon is called tumor progression and, in general, comprises four steps: (1) local overproliferation of cells, (2) extensive overproliferation of cells, (3) invasion of the surrounding tissues and, finally, (4) metastasis, which is the process whereby some malignant cells escape from the original tumor, enter the bloodstream and migrate to other tissue, which they invade and colonize.

Medical practice for PCa has been developed upon the above-mentioned genetic and biological bases as well as on the accumulated experience of physicians treating this disease. The current medical protocols include all these sources of knowledge on PCa in the form of statistics for the probability of cancer stage and treatment success. In brief, PCa is easier to cure in its early stages, before it becomes excessively aggressive and spreads out of the prostate but, unfortunately, this disease hardly ever produces any symptom until the tumor is either very large or has invaded other tissues.

Presently, the best way to combat PCa is a combination of prevention and regular screening for early detection. Regular screening for PCa usually consists of a Prostate Specific Antigen (PSA) test and a Digital Rectal Examination (DRE), which are usually performed periodically in men over age fifty. The PSA test is a blood test for measuring the serum level of this prostate activity biomarker, which rises during PCa. The DRE is a physical test in which the doctor palpates the rectal wall nearby the prostate searching for unusual firm masses, as healthy prostates are normally compliant. If either the DRE or the PSA test is positive, the patient will be asked to undergo a biopsy, an invasive procedure performed with a needle guided by transrectal ultrasound (TRUS), to obtain an average of 8 to 12 tissue cores. If cancerous cells are found in the biopsy, the structure and organization of the aberrant cells will be analyzed by a pathologist to determine the aggressiveness of the tumor, which is measured by a heuristic histopathological indicator known as the Gleason score. With the results of the DRE, the biopsy and some possible medical images, such as Magnetic Resonances (MRs) or Computed Tomographies (CTs), PCa guidelines are used to establish a clinical stage, that is, an estimation of how far cancer has progressed. Taking the clinical stage, PSA level and Gleason score together, protocols are utilized to define the risk group for a patient and associate a series of alternative treatments.

The selection of the treatment, such as surgery (radical prostatectomy), cryoablation, thermal ablation, high intensity focused ultrasound (HIFU), radiation therapy, hormonal therapy, chemotherapy or a combination of them, takes into account age, life expectancy, other aspects of the clinical history, and the personal preferences of individual patients. The chosen treatment may be of a curative or palliative nature, the former being the standard for localized PCa and the latter being the common practice for advanced PCa. In some cases, if the tumor is in its very early stages, or if the risk is judged to be very low, a curative treatment may be delayed while the patient is monitored until the appropriate moment for treatment (active surveillance). Patients that are not eligible for local curative treatment, or those with a short life expectancy, may benefit from a deferred palliative treatment, aimed at tackling specific symptoms of the disease, while they continue on regular screening (watchful waiting).

Biologists and physicians are actively engaged in the study of PCa in an attempt to shed light on the molecular, biological and physiological mechanisms that explain how tumors originate, grow and spread within the body. Despite the number of experimental and clinical investigations, a comprehensive theoretical model into which the abundance of data can be organized and understood is lacking.

### SUMMARY

In one set of embodiments, a method of modeling the possible progression of prostate cancer in a subject may be implemented as follows.

The method includes performing operations on a computer system, wherein the operations include simulating evolution of a tumor in a prostate gland of the subject. The action of simulating is based at least on a coupled system of reaction-diffusion equations and a patient-specific geometric model of the prostate gland of the subject. The term “subject” and the term “patient” are used herein interchangeably.

In some embodiments, the patient-specific geometric model may be designed based on one or more medical images of the prostate gland of the subject.

In some embodiments, the patient-specific geometric model comprises a three-dimensional (or a two-dimensional) tensor-product spline. In these embodiments, the simulation may be performed in an isogeometric fashion.

In some embodiments, the simulation is performed according to a finite element method, or a finite difference method, or a finite volume method, or a mesh-free method, or an immersed method.

In some embodiments, the patient-specific geometric model is constructed based on any discretization method commonly used for constructing geometric models and/or solving partial differential equation mathematical models on geometric models.

In some embodiments, the reaction-diffusion equations include a first equation representing dynamics of a phase field ϕ and a second equation representing dynamics of a nutrient field σ. The value ϕ(x,t) of the phase field ϕ represents an extent to which cells at position x and time t are cancerous. The value σ(x,t) of the nutrient field σ represents a nutrient concentration at position x and time t.

In some embodiments, the simulation includes storing in memory an output dataset representing said evolution of the phase field ϕ and the nutrient field σ on the geometric model over an interval in simulated time. The output data may be used to perform any of a wide variety of applications (or combinations of applications), as described herein.

In some embodiments, the operations performed by the computer system may also includes simulating evolution of a tissue PSA field p based at least on a third equation and said geometric model. The third equation may be of reaction-diffusion type, and include one or more of the following: a term that represents a rate at which healthy cells produce PSA; a term that represents a rate at which cancerous cells produce PSA; and a decay term that represents a natural decay of the tissue PSA. The tissue PSA field may be used to perform any of a variety of applications, as described herein.

In some embodiments, the operations also include: computing a boundary of the tumor based on a level set of the phase field; and displaying the boundary of the tumor via a display device.

### BRIEF DESCRIPTION OF THE DRAWINGS

Advantages of the present invention will become apparent to those skilled in the art with the benefit of the following detailed description of embodiments and upon reference to the accompanying drawings.

FIGS. 1A-D depict the extraction of the geometry of the prostate and initial tumor from CT data of the patient, according to some embodiments.

FIGS. 2A-F illustrate different growth morphologies adopted by initially spheroidal prostatic tumors, according to some embodiments.

FIGS. 3A-F illustrate the prediction of growth of a prostatic adenocarcinoma at tissue scale, according to one embodiment of the computational model and on a patient-specific basis.

FIGS. 4A-F illustrate tissue PSA distribution across the prostate gland at a particular value of simulation time as well as tumor volume evolution and serum PSA history, according to some embodiments.

FIG. 5A depicts a standard-of-care AS protocol for an illustrative patient, according to some embodiments.

FIG. 5B illustrates the changes to the standard-of-care AS protocol after implementing the computational tumor forecasting pipeline presented in this disclosure, according to some embodiments.

FIG. 5C illustrates the main steps in the disclosed computational pipeline for PCa forecasting during AS, according to some embodiments.

FIGS. 6A-6F summarize the model performance to recapitulate PCa growth in AS, according to some embodiments.

FIGS. 7A-D illustrate the model outcomes for four patients, including the tumor geometry within the 3D anatomic model of each patient's prostate and an axial section of the prostate showing the tumor cell density map obtained with the model and from the ADC measurements at the second and third mpMRI sessions.

FIGS. 8A-F summarize the model performance to recapitulate PCa growth between the first and second mpMRI, as well as the model performance in forecasting PCa growth at the date of the third mpMRI.

FIGS. 9A-D further illustrates the model forecasts for the same four patients in FIGS. 7A-7D.

FIGS. 10A-10H show the resulting distribution of this six potential biomarkers in lower-risk PCa versus higher-risk PCa.

FIGS. 11A-11D show the time trajectories of the model-based markers involved in the calculation of the PCa risk classifier (left) as well as the trajectory of the latter (right) for four patients, respectively.

FIGS. 12A and 12B illustrate ADC-N-GS mapping, according to some embodiments.

FIG. 13 depicts a biomechanistic model of PCa growth during AS, according to some embodiments.

Specific embodiments are shown by way of example in the drawings and will be described herein in detail. It should be understood, however, that the drawings and detailed description are not intended to limit the claims to the particular embodiments disclosed, even where only a single embodiment is described with respect to a particular feature. On the contrary, the intention is to cover all modifications, equivalents and alternatives that would be apparent to a person skilled in the art having the benefit of this disclosure. Examples of features provided in the disclosure are intended to be illustrative rather than restrictive unless stated otherwise.

The headings used herein are for organizational purposes only and are not meant to be used to limit the scope of the description. As used throughout this application, the word “may” is used in a permissive sense (i.e., meaning “having the potential to”), rather than the mandatory sense (i.e., meaning “must”). The words “include,” “including,” and “includes” indicate open-ended relationships and therefore mean “including, but not limited to”. Similarly, the words “have,” “having,” and “has” also indicated open-ended relationships, and thus mean “having, but not limited to”. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third fastener coupled to a garment” does not preclude scenarios in which a “fourth fastener coupled to the garment” is connected prior to the third fastener, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task. In some contexts, “configured to” may be a broad recitation of structure generally meaning performs the task or tasks during operation. As such, the component can be configured to perform the task even when the component is not currently on.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The following is a glossary of terms used in this disclosure:
Memory Medium—Any of various types of non-transitory memory devices or storage devices. The term “memory medium” is intended to include an installation medium, e.g., a CD-ROM, floppy disks, or tape device; a computer system memory or random access memory such as DRAM, DDR RAM, SRAM, EDO RAM, Rambus RAM, etc.; a non-volatile memory such as a Flash, magnetic media, e.g., a hard drive, or optical storage; registers, or other similar types of memory elements, etc. The memory medium may include other types of non-transitory memory as well or combinations thereof. In addition, the memory medium may be located in a first computer system in which the programs are executed, or may be located in a second different computer system which connects to the first computer system over a network, such as the Internet. In the latter instance, the second computer system may provide program instructions to the first computer for execution. The term “memory medium” may include two or more memory mediums which may reside in different locations, e.g., in different computer systems that are connected over a network. The memory medium may store program instructions (e.g., embodied as computer programs) that may be executed by one or more processors.
Carrier Medium—a memory medium as described above, as well as a physical transmission medium, such as a bus, network, and/or other physical transmission medium that conveys signals such as electrical, electromagnetic, or digital signals.
Computer System—any of various types of computing or processing systems, including a personal computer system (PC), mainframe computer system, workstation, network appliance, Internet appliance, personal digital assistant (PDA), television system, grid computing system, or other device or combinations of devices. In general, the term “computer system” can be broadly defined to encompass any device (or combination of devices) having at least one processor that executes instructions from a memory medium.
Processing Element—refers to various implementations of digital circuitry that perform a function in a computer system. Additionally, processing element may refer to various implementations of analog or mixed-signal (combination of analog and digital) circuitry that perform a function (or functions) in a computer or computer system. Processing elements include, for example, circuits such as an integrated circuit (IC), ASIC (Application Specific Integrated Circuit), portions or circuits of individual processor cores, entire processor cores, individual processors, programmable hardware devices such as a field programmable gate array (FPGA), and/or larger portions of systems that include multiple processors.
Automatically—refers to an action or operation performed by a computer system (e.g., software executed by the computer system) or device (e.g., circuitry, programmable hardware elements, ASICs, etc.), without user input directly specifying or performing the action or operation. Thus the term “automatically” is in contrast to an operation being manually performed or specified by the user, where the user provides input to directly perform the operation. An automatic procedure may be initiated by input provided by the user, but the subsequent actions that are performed “automatically” are not specified by the user, i.e., are not performed “manually”, where the user specifies each action to perform. For example, a user filling out an electronic form by selecting each field and providing input specifying information (e.g., by typing information, selecting check boxes, radio selections, etc.) is filling out the form manually, even though the computer system must update the form in response to the user actions. The form may be automatically filled out by the computer system where the computer system (e.g., software executing on the computer system) analyzes the fields of the form and fills in the form without any user input specifying the answers to the fields. As indicated above, the user may invoke the automatic filling of the form, but is not involved in the actual filling of the form (e.g., the user is not manually specifying answers to fields but rather they are being automatically completed). The present specification provides various examples of operations being automatically performed in response to actions the user has taken.
Configured to—Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112, paragraph six, interpretation for that component.

Described herein is a phenomenological model for PCa that qualitatively reproduces in vitro experiments and the actual growth patterns of PCa during the early to mid-late stages of the disease, when PCa is usually diagnosed and treated. The computer simulations carried out with the described model suggest that prostate tumors may escape starvation and necrosis by developing a tubular shape or branching.

Although the potential of PSA as a PCa biomarker remains unclear, PCa diagnosis and monitoring in clinical practice relies heavily on PSA time evolution. Thus, in an embodiment, an equation that models the PSA dynamics within the prostatic tissue is described. Under certain assumptions, the integration of this equation leads to another equation that governs the dynamics of serum PSA, whose solution can be directly interpreted by a urologist.

In addition to the diagnosis and treatment of PCa, the PSA model can be used to investigate the role of PSA as a biomarker for PCa, which is currently one of the most contentious debates in the urology community. The herein described modeling and simulation technology also addresses new challenges posed by the tissue-scale problem. Traditional smaller-scale modeling usually ignores the geometry of the host tissue. Yet, experimental evidence shows that tissue geometry determines sites of branching morphogenesis and plays a key role in tissue growth, and, consequently, in cancer development. Accounting for tissue geometry is especially relevant in PCa because prostate anatomy varies significantly from one person to another, and PCa almost invariably develops in the peripheral zone of the prostate, close to the prostatic capsule. Thus, the combination of patient-specific anatomy and PCa modeling provides a model which is customized for the patient. In an embodiment, techniques of computational geometry are used to extract the precise anatomy of the prostate from medical images, as well as the initial location and shape of the tumor for a patient. Fully three-dimensional computer simulations are performed that predict the growth of the tumor, including the first patient-specific simulation of PCa evolution based on prostatic anatomy obtained from medical images.

In an embodiment, the possible progression of prostate cancer in a subject can be modeled by performing operations on a computer system, wherein the operations include: simulating evolution of a tumor in a prostate gland of a subject, wherein said simulating is based at least on a coupled system of reaction-diffusion equations and a geometric model of the prostate gland of the subject. The use of reaction-diffusion equations and a geometric model of the prostate gland of the subject provides a tumor growth model that predicts the expected progression of prostate cancer in the individual. The tumor growth model may be used to devise a customized treatment for the subject.

Most continuous mathematical models of tumor growth developed to date consider several tumor species and the host tissue as different interacting phases that compete in order to obtain nutrients, proliferate and occupy more space so as to thrive in the tumoral environment. Different malignant cell types are associated with different traits, such as whether the cells are alive or not (viable, necrotic), the oxygen concentration in their local environment (normoxic, hypoxic), and whether they exhibit a certain phenotype or acquired behavior due to genetic alterations (e.g., proliferative, invasive, resistance to a certain treatment). The dynamics of each cellular species is usually modeled with a diffusion-reaction equation. In some cases, convection is also accounted for, as well as other types of migration, for example, chemotaxis, that is, cell movement driven by gradients of dissolved substances in the cell environment, or haptotaxis, that is, cell movement driven by gradients of substances attached to the extracellular matrix. The dynamics of the nutrients, as well as any other substance with a major role in cancer progression, is included by means of additional diffusion-reaction equations.

The reaction-diffusion equations include a first equation representing dynamics of a phase field ϕ and a second equation representing dynamics of a nutrient field σ. In an embodiment, a value ϕ(x,t) of the phase field ϕ represents an extent to which cells at position x and time t are cancerous, wherein a value σ(x,t) of the nutrient field σ represents a nutrient concentration at position x and time t. The position x is a vector in two or three dimensions.

In the model presented herein, the phase-field method is used to account for the healthy-to-tumoral transition and the coupled dynamics of both the host tissue and the cancerous cells, resulting in the diffusion-reaction model in equation [1].

$$\frac{\partial ϕ}{\partial t} = λ Δ ϕ - \frac{1}{τ} \frac{d F ( ϕ )}{d ϕ} + χ σ - A ϕ \qquad [ 1 ]$$

The parameter ϕ is a measure of cellular microstructure. This order parameter may vary between a lower limit and an upper limit, e.g., between 0 and 1, or between −1 and 1. Lower values represent healthy tissue while higher values represent an aberrant cell organization, typical of PCa. A boundary of the tumor corresponds to a level set (e.g., ϕ=0.5) of the phase field.

The growth of PCa is driven by a host of hormones, growth factors, and vital nutrients, but for the sake of simplicity, it is assumed that tumor growth chiefly depends on nutrients, in particular, glucose. The model could also incorporate other substances, but their regulatory effect on PCa growth can be interpreted equivalently in terms of glucose. Hence, these ancillary substances affecting tumor growth can be accounted for by modifying the baseline glucose dynamics. Also, it is assumed that convection has a negligible contribution to the transport of glucose, a fact that is consistent with experimental observations. These assumptions lead to the diffusion-reaction model in equation [2].

$$\frac{\partial σ}{\partial t} = ϵΔσ + s - δϕ - γσ \qquad [ 2 ]$$

In equation [1], F(ϕ) may be a double-well potential, a typical function within the phase-field method, that makes possible the stable coexistence of healthy and cancerous cells in the prostate. For example, F(0) may take on the explicit form:

F(ϕ)=16ϕ²(1−ϕ)². [3]

The last two terms in equation [1] account for nutrient-driven growth and apoptosis (i.e., programmed cell death), respectively. It is assumed that the growth rate depends linearly on the local nutrient concentration with the growth rate coefficient χ. The model further assumes that apoptosis follows a linear relation with the population of tumoral cells, A being its rate, as this is the natural response in the prostatic tissue. In equation [2], s is the nutrient supply. On the right-hand side of the equation, the third term represents the consumption of nutrient by the tumoral cells, while the fourth term accounts for the natural decay of nutrient.

The values for the model parameters can be obtained from studies on nutrient transport within tissues and tumor growth. In some embodiments, the following values are used for the parameters: τ=0.01 year; δ=2.75 g/(l·day); and γ=1000 l/year and are substantially constant. Several values are considered for the growth rate χ and the apoptosis rate A, ranging from χ=400 l/(g·day) to χ=700 l/(g·day) and from A=100 l/year to A=700 l/year, in order to recreate the different morphologies of PCa growth during its first stages. To make the problem computationally tractable, λ˜h²/T, where h is the characteristic length scale of the computational mesh and T is the characteristic time scale, which in some embodiments is considered to be 1 year. In accordance with other models of tumor growth, ϵ˜10λ to ϵ˜100λ, which manifests the experimental observation that tumor dynamics is slower than nutrient dynamics.

Nutrient supply is introduced in two different fashions in order to simulate the experimental results regarding the tumor morphology change. Beginning with a homogeneous supply with a value s₁=2.75 g/(l·day), a series of simulations are preformed which show the shape instability that makes an initially rounded tiny mass of aberrant cells grow with a fingered pattern invading the surrounding tissue. Afterwards, the previous value is set to be the base so upon which a mild heterogeneity c is added, where the heterogeneity takes values between −0.2 g/(l·day) and 0.2 g/(l·day). In this case the nutrient supply will be s₂=s₀+c. Making use of an experimental image, the heterogeneity c is determined according to the vascular network depicted in the image, so that the nutrient supply will be maximum, s=2.95 g/(l·day), near the blood vessels and minimum, s=2.55 g/(l·day), at a point sufficiently far from them. Both s₁ and s₂ attempt to represent in vitro conditions, but the latter aims at reproducing a more realistic scenario. However, for the tissue-scale, patient-specific simulations, a more appropriate constant value s₃=2.70 g/(l·day) is used.

An additional equation that models PSA dynamics within the prostate tissue is used, based on a novel measure for PSA: the tissue PSA p, or serum PSA concentration leaked to the bloodstream per unit volume of prostatic tissue. Hence, tissue PSA will allow us to study the spatial distribution of the sources of serum PSA concentration within the gland. Both healthy and cancerous cells secrete PSA, but the latter generally produce PSA at a much higher rate than healthy cells. Cancerous and healthy rates of PSA production per unit volume of prostatic tissue are designated by α_{c} and αₕ, respectively. Similarly to other substances within the prostate, such as the nutrient, it is assumed that the concentration of tissue PSA p diffuses over the prostatic tissue and decays naturally with a rate γₚ, following equation [4].

$$\frac{\partial p}{\partial t} = η Δ p + {α}_{h} ( 1 - ϕ ) + {α}_{c} ϕ - {γ}_{p} p \qquad [ 4 ]$$

Considering that p represents the concentration of serum PSA that leaks to the bloodstream per unit volume, then serum PSA Pₛ can be defined as the integral of the tissue PSA p over the prostatic tissue Ω, that is,

Pₛ=∫_{Ω}pdΩ [5]

If equation [4] is integrated over the tissue region that is being simulated and assuming free-flux boundary conditions, the following ordinary differential equation is obtained:

$$\frac{d {P}_{s}}{d t} = {α}_{h} {V}_{h} + {α}_{c} {V}_{c} - {γ}_{p} {P}_{s} [ m ] \qquad [ 6 ]$$

where Vₕ and V_{c} denote, respectively, the volumes of healthy and cancerous tissue. Using existing data of prostate gland volumes and age-related PSA scores for a cohort of healthy men, respectively, estimates of αₕ and γₚ were determined by means of equation [6], choosing the values αₕ=6.25 (ng/ml)/(cm³·year) and γₚ=100 l/year, respectively. Indeed, the value of γₚ corresponds to a PSA half-life of 2.5 days, which is consistent with the accepted values. As PSA is produced in higher levels in cancerous cells, the first assumption was α_{c}=15αₕ, yet this proportion can be modified to represent different real cases for each patient, also introducing different levels of PCa malignancy. Finally, as both cancerous cells and a higher production of PSA are linked features in PCa, it is assumed that η=λ.

Taking into account PSA dynamics, the action of simulating evolution of a tissue PSA field p may be based at least on a third equation (such as equation [4]) and said geometric model of the prostate gland of the subject, wherein the third equation is of reaction-diffusion type, where the third equation includes one or more of the following: a term that represents a rate at which healthy cells produce PSA; a term that represents a rate at which cancerous cells produce PSA; a decay term that represents a natural decay of the tissue PSA. Specifically, the third equation may include one or more of the following: a term that is proportional to 1−ϕ (i.e., one minus the phase field ϕ); a term that is proportional to the phase field ϕ; a term that is proportional to the negative of the tissue PSA field.

If PSA dynamics are incorporated into the tumor growth model, the tumor growth model can be used to compute an evolution of serum PSA by numerically integrating the tissue PSA field over a volume of the patient-specific geometric model. The results may be presented by displaying a graph of the evolution of the serum PSA via a display device. In an embodiment, the image of the tissue PSA field may be displayed along a cross section of the patient-specific geometric model (e.g., a user specified cross section), wherein the cross section passes through at least a portion of the tumor. In some embodiments, a video of the evolution of the tissue PSA field on a cross section through the patient-specific geometric model may be displayed, wherein the cross section passes through at least a portion of the tumor.

### Numerical Analysis and Simulations

Any of a wide variety of methods may be used to numerically solve the set of equations that define the tumor growth model, e.g., a finite element method, or a finite difference method, or a finite volume method, or any of various mesh-free methods, or any of various immersed methods. The nonlinearity of the set of equations and the complex geometry of both the prostate and the initial tumor in the patient-specific simulations demand efficient methods of resolution in terms of memory and time of computation. In some embodiments, to perform the numerical simulations, algorithms based on the concept of Isogeometric Analysis (IGA) may be employed. IGA is an emergent and cutting-edge technology that can be seen as a generalization of finite element analysis. In IGA, the standard piecewise polynomials used in the finite element method are replaced with richer functions used in computer graphics and computational geometry, such as, for example, B-Splines, Non-Uniform Rational B-Splines (NURBS) and T-Splines. To speed up the calculations and create a technology that facilitates obtaining solutions in a clinically relevant time, dynamic mesh-adaptivity techniques may be employed, e.g., techniques based on the concept of hierarchical refinement and coarsening, which enables the adaptation of the computational mesh dynamically throughout the simulation such that the approximation functions are richer close to the tumor surface, leading to dramatic savings of memory and compute time.

In an embodiment, a patient-specific geometric model of the prostate gland of the subject is designed based on one or more medical images of the prostate gland of the subject (e.g., images such as X-Ray images, CT images, MM images, ultrasound images, or any combination of the foregoing). The patient-specific geometric model may include a three-dimensional tensor-product spline (such as a B-Spline, or a Non-Uniform Rational B-Spline, or a T-Spline). The locations of model space control points of the tensor-product spline may be specified by a set of user inputs. (For example, the set of user inputs may be received by a computer-aided design (CAD) program executing on a computer system.) The locations of the control points may be specified so that the spline conforms to the subject's prostate gland, as represented in the one or more medical images. The locations of the control points may be specified prior to simulating the evolution of a tumor in a prostate gland. In a further embodiment, the simulation of the evolution of a tumor in the prostate gland is performed in an isogeometric fashion, i.e., using geometric basis functions of the geometric model as analysis basis functions for said simulating.

More generally, the patient-specific geometric model of the prostate gland may be constructed based on any discretization method, e.g., any discretization method known (or commonly used) for solving partial differential equation mathematical models on geometric models.

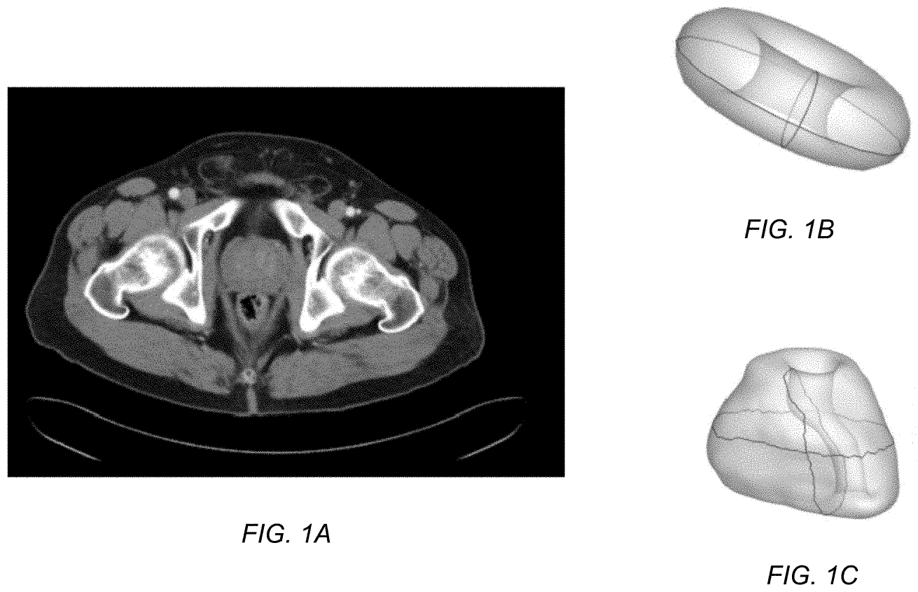

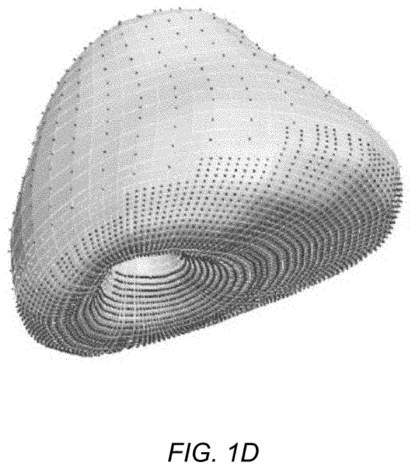

To extract geometric models of the prostate and the initial tumor, several methods may be used to construct solid anatomic NURBS models. Since the geometry of a prostate is topologically equivalent to that of a torus, a parametric mapping method may be used to deform a torus NURBS model to match with the patient-specific prostate surface obtained from CT data, as depicted in FIGS. 1A-D. (NURBS is an acronym for Non-Uniform Rational B-Spline.) Finally, the computational mesh can be hierarchically refined so that it will represent the anatomy of the prostate to perform precise patient-specific simulations.

FIGS. 1A-D depict the extraction of the geometry of the prostate and the initial tumor from CT data. The process to perform tissue-scale, patient-specific simulations may begin by extracting in vivo data of the patient from medical images, such as his prostate anatomy, the tumor location and its shape. FIG. 1A depicts an axial CT image of the patient's abdomen at the height of the middle prostatic gland. As illustrated in FIG. 1B, a NURBS model of a torus is generated. The torus is topologically identical to a prostate. In FIG. 1C, the torus is mapped using computer graphics techniques to the actual geometry of the patient's prostate (posterior view). Finally, in FIG. 1D, hierarchical refinement is used to generate the computational mesh (anterior view).

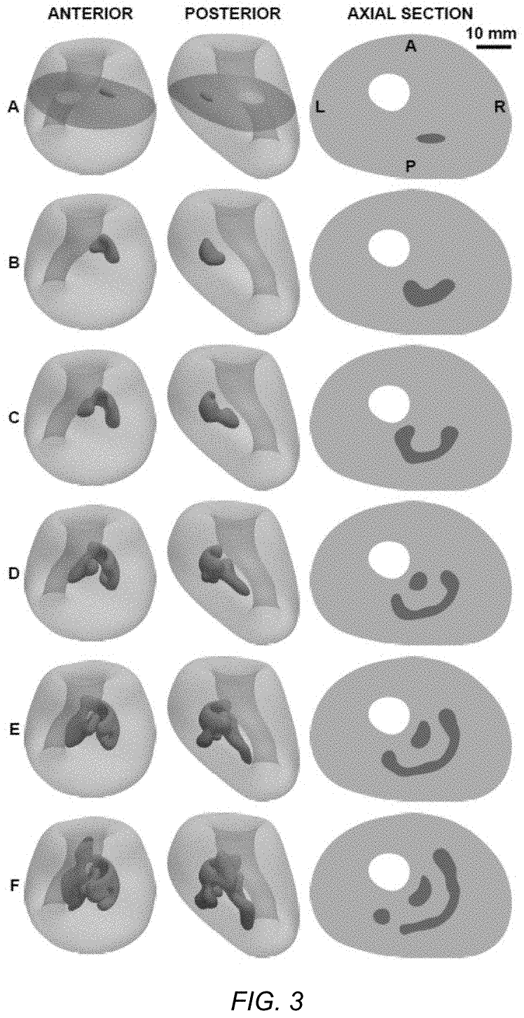

In some embodiments, CT imaging may not be sufficiently accurate to detect localized PCa. Thus, multiparametric Mill data may be used to get the location and estimated shape of the tumor. Alternatively, in order to introduce the initial tumor in the prostate model, the tumor geometry may be approximated as an ellipsoid. In particular, a three-dimensional field (e.g., a hyperbolic tangent function, an inverse tangent function, or any function with similar mathematical properties) may be L²-projected onto the spline space that defines the anatomy of the patient's prostate in order to approximate the initial tumor with the phase field, hence obtaining an ellipsoidal cancerous mass in terms of the order parameter ϕ (see FIG. 3A).

### Growth Patterns of Localized PCa

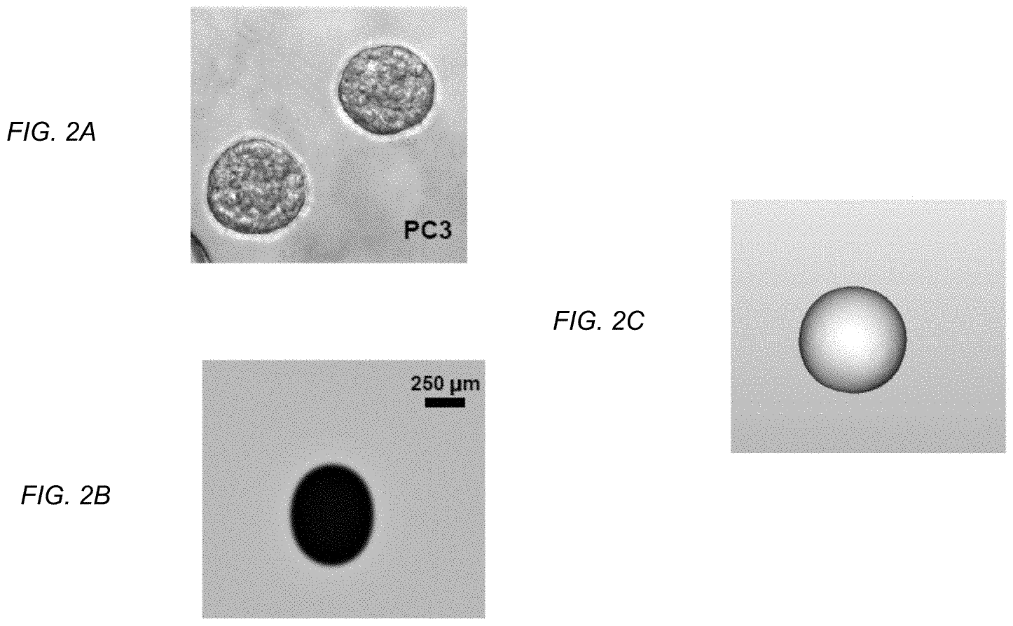

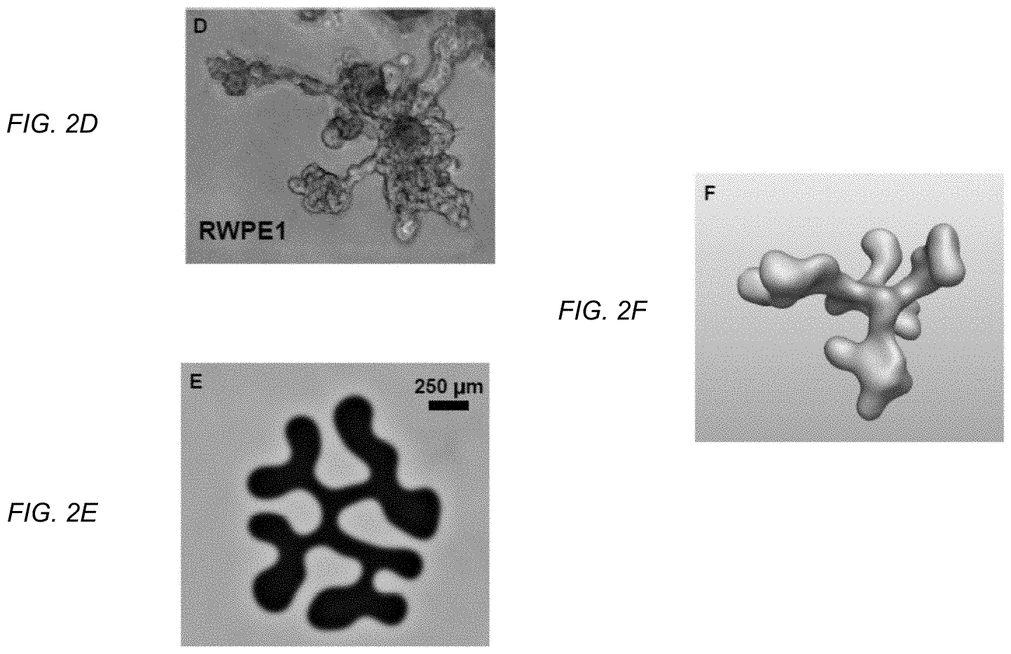

The morphology of PCa may vary from spheroidal tumors to tubular or fingered structures that invade the prostatic epithelium forming branches, as demonstrated by clinical practice and in vitro experimental studies involving cultures of prostate epithelial cells. Large-scale, two-dimensional and three-dimensional simulations of equations [1] and [2] show that the model can predict both growth types (spherical and fingered), as depicted in FIGS. 2A-F. Thus, the model qualitatively reproduces the associated clinical and experimental results for both morphologies.

FIGS. 2A-F illustrate different growth morphologies adopted by initially spheroidal prostatic tumors. Large-scale, two-dimensional and three-dimensional simulations of the model reproduce the spheroidal and fingered tumor growth patterns as seen in clinical practice and experiments (λ=5·10⁻¹¹ cm²/s, ϵ=2·10⁻⁹ cm²/s). The heterogeneous nutrient supply s₂ was used in these simulations to produce more realistic patterns of growth. FIG. 2A depicts in vitro three dimensional Matrigel culture of PC-3 cells growing with a spherical or rounded pattern. Large-scale, two-dimensional (FIG. 2B) and three-dimensional (FIG. 2C) simulations of the model reproducing spheroidal growth (χ=400 l/(g·year), A=300 l/year) are shown. FIG. 2D depicts in vitro three dimensional Matrigel culture of RWPE-1 cells showing a fingered morphology. Large-scale, two-dimensional (FIG. 2E) and three-dimensional (FIG. 2F) simulations of the model reproducing fingered growth (χ=600 l/(g·year), A=600 l/year) are shown.

It has been suggested that this change in tumor morphology is promoted by a series of chemical signals, also motivated by specific mutations or epigenetic changes. However, a conclusive explanation of the mechanisms of this shift in cancer growth remains largely unknown. Shape instability was activated using two mechanisms previously suggested in the literature: first, increasing the parameters χ and A in equation [1], which represents a more aggressive cancer with larger apoptosis, and second, reducing the value of the nutrient supply s, which may be understood as a situation in which the tumor is surrounded by a harsh microenvironment.

Spheroidal or ellipsoidal growth takes place in the very first stages of PCa, when it is not excessively malignant. At these early stages the tumor will feature low values for the growth rate χ and the apoptosis rate A. In particular, taking the aforementioned default values for all the parameters in the model, χ=400 l/(g·year) and A=300 l/year, leads to a spheroidal pattern of growth, as can be seen in FIGS. 2B and 2C. Conversely, the fingered morphology corresponds to a more advanced cancer in comparison to the spheroidal pattern, still within the scope of localized PCa. This tubular growth can be reproduced by the model with higher values of these two parameters, which may also be balanced, that is, they may take similar values. In order to perform the simulations depicted in FIGS. 2E-F, the following assumptions were made χ=600 l/(g·year) and A=600 l/year, which appear to yield a characteristic fingered morphology.

Interestingly, with this latter selection of parameters spheroidal growth was initially observed. Should the tumor continue to develop with this morphology, the nutrient concentration in its central region would drop to values promoting hypoxia, starvation and necrosis. However, once the tumor reaches a volume that would lead to this inner harmful environment, the shift towards the fingered morphology takes place. With this growth pattern, the nutrient concentration within the tumor is not critical any longer. Regardless of whether the homogeneous or the heterogeneous definition for the nutrient supply is used, as long as s₁=s₀, the shape instability will occur at a similar time. However, the spatial distribution will be different, as the homogeneous nutrient supply produces symmetrical patterns of fingered growth and the introduction of a mild heterogeneity promotes growth along the gradients of σ.

If the value of the nutrient supply is reduced, that is, s₁ for the homogeneous version, and so for the heterogeneous one, then fingered growth may take place for a selection of χ and A which would produce spheroidal growth otherwise. Furthermore, in the range of values for χ and A typical of fingered growth, reducing s accelerates the onset of the shape instability. It was also observed that a drop in the values of s leads to slower tumor growth, while increasing the nutrient supply makes the tumor grow at a faster rate. In the case of the fingered morphology, the higher the value of s, the thicker the branches of the tumor, as the resulting higher concentrations of the nutrient within the tissue support larger tumor structures.

In some embodiments, simulating the evolution of a tumor in a prostate gland includes storing in memory an output dataset representing said evolution of the phase field ϕ and the nutrient field σ on the patient-specific geometric model over an interval in simulated time. The output dataset may be used in many different ways, e.g., to study tumor progression.

In one embodiment, the output dataset may be used to estimate a real-world time (e.g., a calendar date) at which one or more of the following tumor events may occur:
(1) the tumor will penetrate a capsule of the prostate gland of the subject;
(2) the tumor will invade a tissue outside (or near) the prostate gland of the subject;
(3) the tumor will reach a specified part of the prostate gland of the subject;
(4) the tumor will transition from ellipsoidal growth to fingered growth; and
(5) the tumor will make contact with the urethra of the subject.
   The real-world time or an indication of the real-world time may be outputted, e.g., to a healthcare professional, via an output device (such as a display device or speaker).

In one embodiment, a video may be generated based on at least a portion of the output dataset, wherein the video illustrates evolution of a boundary of the tumor over time. The video may be outputted, e.g., to a healthcare professional, via an output device (such as a display device).

In one embodiment, an image dataset may be generated based on the output. The image may illustrate a cross section through the patient-specific geometric model and include one or more curves corresponding to (or representing) a boundary of the tumor. The image may be outputted, e.g., to a healthcare professional, via an output device (such as a display device).

In one embodiment, a 3D rendering of the prostate gland and the tumor may be generated (e.g., using the techniques of 3D computer graphics) based at least on the output data set and the patient-specific geometric model. The 3D rendering may be outputted, e.g., to a healthcare professional, via an output device (such as a display device).

In one embodiment, an estimate of a stage of prostate cancer development in the subject may be generated based on the output dataset and multiparametric MM data of the prostate gland of the subject. For example, the estimate may be determined based on a correlation between a tumor boundary in the multiparametric MM image data and a tumor boundary derived from the output dataset. The estimated stage of prostate cancer development may be outputted, e.g., to a healthcare professional, via an output device (such as a display device).

In one embodiment, the output data set may be used to compute a boundary of the tumor based on a level set of the phase field. The computed boundary of the tumor may be outputted, e.g., to a healthcare professional, via an output device (such as a display device). In some embodiments, the volume of the tumor may be determined based on the computed boundary. An indication (e.g., a numerical value or a graph) of the computed volume may be outputted, e.g., to a healthcare professional, via an output device (such as a display device). In some embodiments, a three-dimensional field (e.g., a hyperbolic tangent function or any function with similar mathematical properties) may projected onto a model space corresponding to the geometric model, to approximate the initial tumor with the phase field.

In one embodiment, a health risk (such as prostatic capsule penetration, invasion of nearby tissues or tumoral expansion to different parts of the prostate gland) associated with growth of the tumor in the subject may be predicted based on the output dataset. The health risk determination may be outputted, e.g., to a healthcare professional, via an output device (such as a display device).

In one embodiment, a likelihood (or expected time of occurrence) of an adverse symptom (such as impotence or irregularity in urination and/or ejaculation) associated with growth of the tumor in the subject may be predicted based on the output dataset. An indication of the likelihood (or the expected time of occurrence) of the adverse symptom may be outputted, e.g., to a healthcare professional, via an output device (such as a display device).

In one embodiment, a type of prostate cancer treatment (e.g., chemotherapy, radiation, surgery, high intensity focused ultrasound (HIFU), hormonal therapy, etc.) for the subject may be selected based on the output dataset. An indication of the selected type may be outputted via an output device (e.g., displayed via a display device), e.g., to a healthcare professional.

In one embodiment, a time to start prostate cancer treatment on the subject may be selected based on the output dataset. An indication of the selected time may be outputted via an output device (e.g., displayed via a display device), e.g., to a healthcare professional.

In one embodiment, an anticancer drug or hormone to be administered to the subject may be selected based on the output dataset. An indication of the selected drug or hormone may be outputted via an output device (e.g., displayed via a display device), e.g., to a healthcare professional.

In one embodiment, a session of radiation treatment in order to radiatively kill cells in at least a portion of the tumor may be planned based on the output dataset. For example, a trajectory of beam direction and intensity for a radiation beam may be determined based on the output dataset. A plan for the session may be outputted via an output device (e.g., displayed via a display device).

In one embodiment, a chemotherapy treatment may be planned based on the output dataset, in order to increase the effectiveness of the treatment. A chemotherapy treatment plan may be outputted via an output device (e.g., displayed via a display device).

In one embodiment, a session of high intensity focused ultrasound (HIFU) may be planned based on the output dataset, in order to kill cells in at least a portion of the tumor. A plan for the HIFU session may be outputted via an output device (e.g., displayed via a display device).

In one embodiment, a session of surgery may be planned based on the output dataset, e.g., surgery to remove at least a portion of the tumor. A surgery plan may be outputted via an output device (e.g., displayed via a display device), e.g., to a health professional. The surgery plan may include, e.g., a specification of a series of surgical incisions.

In one embodiment, a robotic surgical tool (e.g., scalpel) may be controlled based on the output dataset, in order to remove at least a portion of the tumor.

In one embodiment, the output dataset may be used to determine invasive pathways for sampling and/or administration of therapeutic agents. For example, an insertion path of a biopsy needle may be determined based on the output data set, in order to increase a likelihood of obtaining samples of tumor tissue. An insertion path of a syringe may be determined based on the output dataset in order to inject cancer killing chemicals into at least a portion of the tumor. An insertion path of a syringe may be determined based on the output dataset in order to sample fluid and/or cells from at least a portion of the tumor. The proposed invasive pathway may be outputted, e.g., to a healthcare professional, via an output device (such as a display device).

The phrase “outputting via an output device” may take various forms in various embodiments. In some embodiments, the output device may include a display device, in which case the action of “outputting” includes displaying via the display device. In other embodiments, the output device may include a speaker, in which case the action of “outputting” includes outputting an audio signal via the speaker. In other embodiments, the output device may include a transmitter, in which case the action of “outputting” includes transmission of a signal (or information) over a transmission medium, e.g., a wireless medium or a wired medium. For example, the outputting may include transmission of information over the Internet or other computer network. In some embodiments, the output device may include a 3D printer, in which case the action of “outputting” includes sending information to the 3D printer in order to print a 3D object, e.g., an object representing the tumor (or the prostate gland including the tumor).

### EXAMPLES

### Patient-Specific Simulation

The Austin Radiological Association provided us with a series of axial and coronal CT images of a small cohort of patients with PCa, with and without contrast. CT is a not a suitable technique for the detection of localized PCa due to its reduced contrast between healthy and cancerous tissue within this gland. However, it is implemented in PCa protocols primarily in order to scan for metastasis or as a resource to initially analyze the prostate anatomy and the pelvic region with more detail than ultrasound.

A patient was chosen in order to predict the evolution of his prostatic tumor using the model described herein and assess the performance of the simulation on the basis of the current knowledge of PCa evolution. This patient showed a small cancerous lesion of 0.03 cm³, previously detected by ultrasound, in the posterior part of the peripheral zone at the middle region of the prostatic gland. The volume of the prostate gland is considered constant along the simulation and has a value of 71.67 cm³. FIGS. 3A-F represent the prediction of the growth of a prostatic adenocarcinoma according to the model at tissue scale and on a patient-specific basis. As shown in FIGS. 3A-F, the model was run on the actual geometry of the prostate of the patient under consideration, after extracting it and the initial tumoral volume from the CT output files. These images show the predicted evolution of a prostatic tumor on the actual anatomy of the prostate of an individual patient (λ=0.24 mm²/day, ϵ=7.5 mm²/day). The axial section depicted is at z=25.5 mm, with the origin of the coordinate z set at the prostate base (A: anterior, P: posterior, L: patient's left, R: patient's right). The position of this section has been shaded in the anterior and posterior views for t=0. FIG. 3A depicts the initial tumor as identified in the patient's CT images. FIGS. 3B-F depict predicted growth according to the model during 1 year, with each sub figure corresponding to a time of 0.2 years apart.

This simulation represents tumor growth during 1 year and at the end the tumor volume is 2.66 cm³. Initially, the tumor grows following an ellipsoidal pattern but it soon starts to develop preferential directions of growth, defining finger-like structures. As the tumor expands within the prostate, its geometry evolves toward a structure consisting of thickened layers of cancerous tissue with fingered protrusions along the main directions of growth. The axial section at the mid-gland shows an evolution from the spheroidal pattern of growth to the fingered morphology similar to that observed in large-scale, 2D and 3D simulations. The tumoral area in these sections qualitatively matches some delineations of PCa made on actual histopathologic specimens.

### Tissue and Serum PSA

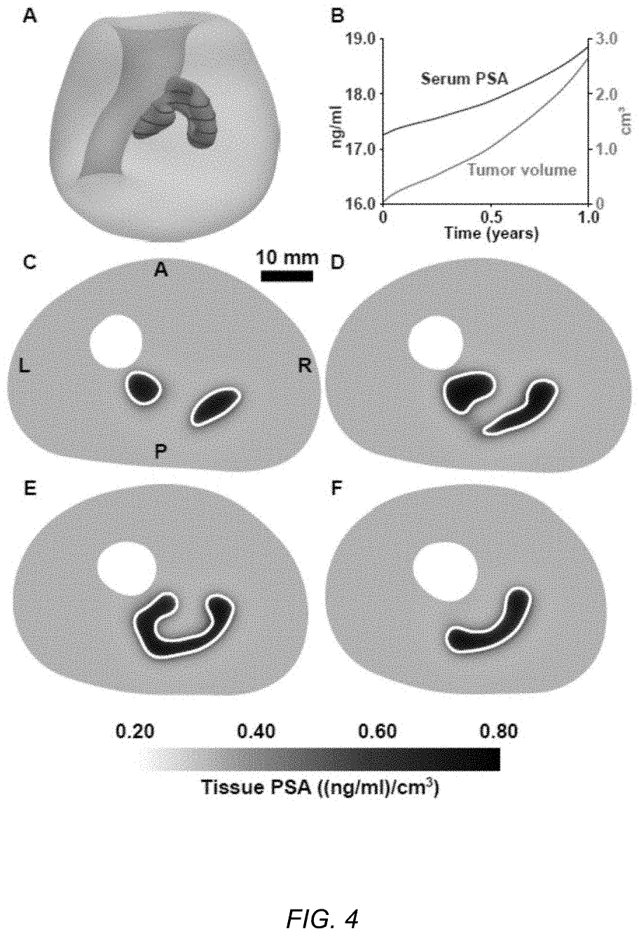

In the simulations, tissue PSA evolution is obtained from equation [4], and serum PSA evolution is then obtained from equation [6]. The field of tissue PSA typically shows higher values in the central areas of the tumor and lower values further away from the lesion, as depicted in FIGS. 4A-F. The difference in the values of p in healthy and cancerous tissue is due to the fact that the initial tumor starts producing PSA at a higher rate from the first moment and, as the tumor grows, higher tissue PSA values are found in the areas occupied by cancerous cells and following the preferential directions of growth. In general, tissue PSA values do not differ greatly between healthy and tumoral tissue in the model's 2D and 3D experimental simulations. However, in the tissue-scale, patient-specific simulations, the values of p in the tumor, around 0.70 (ng/ml)/cm³, were an average of 3 times higher than in the healthy regions, around 0.24 (ng/ml)/cm³, as seen in FIGS. 4A-F.

Taking lower values for α_{c}, but greater than αₕ, the field of tissue PSA becomes more homogeneous, meaning that the peak values are lower too, due to the reduced difference between the rates of production of PSA in healthy and cancerous cells. The evolution of serum PSA is linked to the velocity of tumor growth. The faster the tumor grows, the faster serum PSA increases because there are more tumoral cells, producing PSA at a higher rate. Taking lower values for α_{c} also induces lower PSA scores in blood and the PSA velocity is also slowed down.

The serum PSA is obtained by integrating the values of p over the entire prostate anatomy. Since the biochemical data for the patients was not available, the baseline serum PSA was estimated according to the tumor stage and the dynamics imposed by the model. Beginning with the minimum value of 4.0 ng/ml, beyond which a physician would recommend a TRUS-guided biopsy, a steep jump towards a higher value was observed in the first time steps and then serum PSA followed exponential dynamics, as observed in clinical practice. Therefore, in order to produce more realistic simulations, the model was restarted once this readjustment of serum PSA was completed, correcting the initial condition for p so that it corresponded to the value of serum PSA reached just after the rising branch. Hence, serum PSA readjustment was eliminated and it followed exponential dynamics from the beginning of the simulations. In particular, an initial serum PSA level of 17.3 ng/ml was estimated and, at the end of the simulation, PSA reaches 18.9 ng/ml.

In FIGS. 4A-F, tissue PSA distribution across the prostate gland at t=0.5 years, tumor volume evolution and serum PSA history are depicted. Tissue PSA takes higher values on the tumoral tissue because it is produced by cancerous cells at a higher rate. FIG. 4A depicts an anterior view of the prostate and tumor at t=0.5 years with the location of the axial sections depicted in this figure as black contours on the tumor. FIG. 4B depicts predicted serum PSA and tumor volume time histories. FIGS. 4C-F depict evenly-spaced axial sections of the prostate representing the distribution of tissue PSA p at time t=0.5 years and height z=18.3 mm, z=21.6 mm, z=24.9 mm, and z=28.2 mm, respectively, with the origin of the coordinate z set at the prostate base. The white line is the contour of the tumor (A: anterior, P: posterior, L: patient's left, R: patient's right).

Predictive medicine and mathematical oncology offer a cutting-edge approach to medical practice. These new trends may be the key to organize and broaden the understanding of some critical diseases nowadays, such as PCa, by providing robust, comprehensive theoretical models integrating all the major known and forthcoming results from research in biological, biophysical, medical and biomedical sciences as well as engineering and computational mechanics. In particular, the simple model presented herein is able to reproduce the typical features of prostatic adenocarcinoma growth during the first to mid-late stages of this disease as seen in experimental results and clinical practice. This model may be implemented in clinical software for physicians to aid in the diagnosis of the disease, the prediction of its evolution, assessment of alternative treatments, and management of follow-up.

### Tissue-Scale, Patient-Specific Simulations

Patient-specific simulations (see FIGS. 3A-F) were carried out over the actual anatomy of a patient, introducing the tumor morphology at the time of diagnosis by extracting both geometries from CT output data. It was observed that if the position of the tumor was changed within the prostate, but conserved its original shape, the tumoral pattern of growth was different. These results suggest that cancer growth morphology is dependent on the particular geometry of the tumor as well as on the specific anatomy of the prostate. Consequently, this observation supports the major importance of considering patient-specific anatomies for both the prostate and the tumor to accurately predict tumor growth. Introducing the particular anatomy of a patient in the model is crucial in order to predict the potential risks that PCa growth may entail, such as prostatic capsule penetration, invasion of nearby tissues or tumoral expansion to the different parts of the prostate, hence triggering symptoms such as impotence or irregularities in urination and ejaculation.

The possibility to predict when these risks or symptoms will appear would be a major advance in watchful waiting and active surveillance. Hence, the optimization of these two clinical options to manage the disease, including the modeling and simulation of PCa, would decrease the overtreatment of patients, in many instances leaving them relieved or cured after the delivery of the corresponding treatment, with minimum side effects and a good quality of life. The inclusion of the individual's prostate and tumor geometries is important in order to adequately follow up the progression of the disease in terms of tumor volume and location and PSA production, both before and after the delivery of a certain treatment. Predicting the actual tumor shape and volume within the anatomy of the prostate of a given patient would enable optimization of surgery and radiation therapy planning and delivery, obtaining high precision and efficiency rates.

The simulation presented in FIGS. 3A-F shows an important and typical feature of PCa, namely, why some biopsies are falsely negative or apparently yield a lower tumoral stage. As the tumor grows, it develops finger-like structures after the morphology shift that, in a three-dimensional, patient-specific scenario, evolve not only as branches but also as thickened layers of cancerous cells. Likewise, there are other layers and regions of healthy tissue surrounding and in between these cancerous layers, which are required in order to provide an adequate distribution of the nutrient. When a biopsy is performed following the current standards, a number of tissue samples are obtained in prescribed positions trying to map the whole prostatic gland. However, the tumor might be out of the reach of the needle used in the biopsy, resulting in a false negative, or it may target one of these tumoral thickened layers orthogonally, what may produce a low percentage of tumor in the tissue sample, leading to further biopsies or/and an erroneous stage of PCa that may compromise the patient quality of life and survival. One might conclude that the pathology analysis of a prostatic tumor biopsy would always provide a lower bound to the severity of the cancer, creating a dangerous predictive scenario.

Ideally, the combination of multiparametric MRI and computational modeling and simulation of PCa may eliminate the biopsy of the prostate during PCa diagnosis and staging, hence dropping an invasive procedure with questionable outcomes and under continuous debate in the medical community. In fact, the combination of T2-weighted MRI, diffusion-weighted MRI, dynamic contrast-enhanced MM and, optionally, magnetic resonance spectroscopic imaging, has already been able to detect and stage PCa with high sensitivity and specificity, comparable to those of current standard biopsies. As a result, multiparametric MRI is now increasingly regarded as a future option to biopsy or, at least, a technique to be fused to TRUS-guided biopsy in order to optimize this procedure.

### Modeling and Simulation of PSA Kinetics

An equation to model the behavior of PSA may also be added by introducing a novel measure, tissue PSA or the serum PSA concentration leaked to the bloodstream per unit volume of prostate tissue, equation [4]. This can be integrated over the computational domain that represents the anatomy of the prostate, allowing serum PSA dynamics to be followed as shown in equation [6]. The tissue PSA allows where PSA is being leaked to the bloodstream to be analyzed and the amount of this PCa biomarker discharged by each type of tissue, as depicted in FIGS. 4A-F. Furthermore, the results presented in FIGS. 4A-F also show that the model is able to compute a realistic evolution for serum PSA, similar to those seen in the literature. Tissue PSA links serum PSA to tumor burden in the model, hence establishing a theoretical basis for the link between both dynamics. A corroboration of this connection arose when the baseline serum PSA was estimated for the simulation. For a given selection of parameters in equations [1] and [4], and the initial tumoral volume, the initial serum PSA was intrinsically determined. Therefore, if the simulation begins with a different serum PSA value, a steep branch would appear until it was readjusted. Additionally, it can be seen in FIGS. 4A-F that the evolution of the tumoral volume and that of serum PSA follow similar dynamics. This behavior is typically seen in low- and intermediate-risk tumors. Nonetheless, the connection between tumor growth and serum PSA dynamics is an ongoing major debate in the urologic community. Regular screening of PCa relies on PSA tests but, even though most prostatic adenocarcinomas grow showing a parallelism between the tumor volume and PSA levels in blood, it is known that there exists a certain degree of divergence between the dynamics of these two variables. This may even reach the limit of patients with low PSA and high tumoral masses in their prostates, or vice versa. Advanced and metastatic PCa may also exhibit almost no connection between serum PSA and tumor burden. However, PSA screening has greatly improved the early detection and treatment of PCa, leading to higher rates of incidence and an improved efficiency in its management worldwide. This biomarker has, therefore, been implemented in a mathematical model of PCa, as it permits direct connection with existing clinical data.

The model described herein may be expanded and improved in many different ways. First of all, several cancerous species could be considered, for instance, normoxic, hypoxic and necrotic, so as to better reproduce the shape instability. The model could also account for other substances that are known to have a role in PCa, such as oxygen or testosterone. Haptotaxis could play a role in more aggressive PCa and the invasiveness of PCa could be better reproduced if the diffusive term in equation [1] is defined in a more sophisticated fashion. PSA modeling in the medical community may be worth revisiting in order to find further connections between tumor shape and volume and PSA production, hence improving the definition of the rates αₕ and α_{c}, or suggesting a refinement of equation [4]. Modeling and simulation of the effects of radiation therapy in PCa may also be incorporated into the model, following previous studies carried out for low grade gliomas and glioblastoma multiforme.

Implementation of Mechanics into Modeling and Simulation of Tumor Evolution

Various embodiments may be contemplated that implement mechanics into the modeling and simulation methods of the present disclosure. Implementation of mechanics into the disclosed modeling and simulation methods may further refine the models and simulations to increase their accuracy and/or provide additional functions or outputs. Mechanics may be implemented, for example, by addition of various equations that describe mechanics associated with tumor growth. Additionally, equations related to various treatment options (e.g., drug therapy or treatment protocols) may also be implemented to provide advantageous results.

In certain embodiments, mechanics equations implemented include equations of mechanical equilibrium in the prostate gland. In such equations, one term may represent the stresses induced by tumor growth. This term may be a function of a variable representing the position of the tumor tissue (e.g., a position x of the tumor tissue at a time t). Another term may represent the stresses induced by benign prostatic hyperplasia. This term may be defined over the central gland of the prostate and be a function of the growth rate of the prostate central gland and time. The growth rate of the prostate central gland may, in some embodiments, be assumed to be constant over the central gland. In other embodiments, the growth rate of the prostate central gland may be assumed to be a heterogeneous map constructed upon imaging data. For instance, the heterogenous map may be implemented to account for the position of hyperplastic nodules with varying developing rates in the prostate central gland. The implementation of equations of mechanical equilibrium may be useful in accounting for mechanical deformation of the prostate due to coexisting prostate cancer (e.g., tumor growth) and benign prostatic hyperplasia.

In various embodiments, the mechanical properties of prostatic tissue are defined for each internal radiological region of the prostate gland. For example, different mechanical properties can be defined for the peripheral zone and the central gland of the prostate. In some embodiments, a heterogenous field definition based on imaging data may be implemented to define the mechanical properties of the prostatic tissue. In some embodiments, the mechanical properties of the prostatic tissue may vary over time, for example as the prostate undergoes deformation due to tumor growth and/or developing benign prostatic hyperplasia. In some embodiments, the relative proportion of stromal and epithelial tissue from each region of the prostate (e.g., measured from biopsy samples or estimated from imaging data) may be implemented to define the mechanical properties of the prostatic tissue. In some instances, the heterogenous field definition, the time-varying definition, and the relative proportion implementations may be combined to define the mechanical properties of the prostatic tissue.

In various instances, the boundary conditions on the external surface of the prostate have a Winkler-inspired formulation. In this formulation, the mechanical tractions at each point of the outer surface of the prostate are negatively proportional to the displacements at said point. In some embodiments, the proportionality constants are assumed equal or different in three directions of space. Additionally, the proportionality constants may vary according to the different tissue types in contact with the exterior surface of the prostate.

In certain embodiments, the mechanical stress field at the initial time of the model includes an estimation of the mechanical stresses introduced by benign prostatic hyperplasia before the time of the first imaging dataset used to initialize the model. This estimation may be obtained by solving an inverse problem using the mechanical equilibrium equations in the prostate only implemented with the benign prostatic hyperplasia term described above (e.g., the tumor term described above is not implemented). Solving this estimation may be implemented to assess the benign prostatic hyperplasia growth rate and proportionality constants in the boundary condition that best match an atlas-based geometry of the prostate at age 40 and the patient's prostate geometry in the imaging dataset used to inform the model. In certain embodiments, when two or more imaging datasets have been collected for a patient, they can be leveraged to solve the aforementioned inverse problem instead of atlas-based geometry.

In certain embodiments, the mechanics equations implemented include equations of mechanotransductive function in the prostate gland. Equations of mechanotransductive function may adjust for the mobility and net proliferation in the tumor dynamics equation, described above. In some embodiments, the mechanotransductive function depends on a scalar metric or a group of scalar metrics that summarize the mechanical stress state in each point of the patient's prostate. For example, von Mises stress and the hydrostatic stress state may be included in the mechanotransductive function. Placing the mechanotransductive function in the tumor dynamics equation reduces the mobility and net proliferation in the equation as the local mechanical stress state intensifies and as measured through the metrics described. Net proliferation is the difference between proliferation and cell death processes in the tumor dynamics equation). As described above, implementation of the mechanotransductive function may be useful in assessing the effects of mechanical stresses on tumor dynamics.

In various embodiments, the patient's response (e.g., the prostate gland response) to a therapy may be assessed using the mechanics equations in the tumor growth model/simulation. The assessment may be made in instances where the patient is already undergoing therapy or also in instances where the patient is being evaluated to start the therapy. In some contemplated embodiments, the therapy includes the use of 5-alpha reductase inhibitor drugs.

For the assessment of a therapy based on 5-alpha reductase inhibitors, one or more terms that represent the net proliferation of tumor cells in the tumor dynamics equation may be adjusted with a drug therapy adjustment factor. The drug therapy adjustment factor may be, for example, a factor that represents an increase in drug-induced tumor cell death. Implementation of this factor may result in reduced tumor cell net proliferation. In some embodiments, this factor may be defined as a constant over the whole prostate gland. In some embodiments, this factor may be varied over the prostate gland and over time based on patient-specific imaging or biopsy-collected histopathological data.

In various embodiments for the assessment of a therapy based on 5-alpha reductase inhibitors, the equation(s) of mechanical equilibrium may feature an additional term that represents the inhibition of benign prostatic hyperplasia and the drug-induced shrinkage of the prostate. Parameterization of this additional term may be implemented using population data from the literature or calibrated using patient-specific longitudinal data collected during the drug therapy (e.g., during treatment with 5-alpha reductase inhibitors).

As described above, the effects of 5-alpha reductase inhibitors may be assessed utilizing mechanics equations. This assessment may be useful in evaluating whether treatment with 5-alpha reductase inhibitors may result in a patient's radiologically and histopathologically-confirmed tumor exhibiting faster tumor dynamics, which may be indicative of increased malignancy. In some embodiments, the assessment of 5-alpha reductase inhibitors may be useful in evaluating whether treatment with 5-alpha reductase inhibitors may result in a patient's radiologically detected prostatic lesion, which is suspicious of being a tumor, is exhibiting faster dynamics indicative of increased malignancy.

In various embodiments, a therapeutic plan based on 5-alpha reductase inhibitors for a patient may be developed based on assessment of the effects of these drugs using the model. For instance, a therapeutic plan may be developed that maximizes prostate shrinkage while minimizing the development of the patient's prostatic tumor or radiologically-detected prostatic lesion suspected of being a tumor. Such a therapeutic plan may provide more optimized treatment of a patient than developing a therapeutic plan without this information.

In some embodiments, assessment of the mechanics equations in the model/simulation of tumor growth may be utilized to determine the timing of imaging scans, tumor biopsies, or PSA tests during treatment/monitoring of a patient's health. For instance, the timing of imaging scans and/or tumor biopsies may be developed based on information assessed in the absence of treatment, during treatment, and/or after treatment. The assessment may be made, as described above, on a patient's existing tumor or radiologically-detected prostatic lesion suspected of being a tumor. In particular, the assessment may be made for a therapy based on 5-alpha reductase inhibitors.

In some embodiments, assessment of the mechanics equations in the model simulations may be utilized to perform longitudinal, non-rigid registration of imaging data of the prostate for individual patients. In some embodiments, assessment of the mechanics equations in the model simulations may be utilized to assess the progress of the benign prostatic hyperplasia and/or develop treatment plans for benign prostatic hyperplasia. Assessment of the mechanics equations in the model simulations may further be utilized to assess the progression of prostate cancer during the monitoring of the patient (e.g., during active surveillance, or during/after primary treatment for the tumor(s) in the prostate) and/or develop treatment plans for the prostate cancer. For example, treatment plans involving surgery or radiotherapy may be developed based on the assessment of the mechanics equations in the model simulations In some embodiments, assessment of the mechanics equations in the model simulations may further be utilized to plan biopsies of the prostate.

In certain instances, an abnormal deformation of the prostate may be indicative of a growing prostatic tumor. In such instances, assessment of the mechanics equations in the model simulations may be utilized to detect the abnormal deformation. Additionally, the assessment may be implemented to guide the performance of an ensuing biopsy to confirm the diagnosed pathology of the abnormal deformation.

Implementation of MRI-Based Forecasting into Modeling and Simulation of Tumor Evolution

As discussed herein, prostate cancer (PCa) is a public health burden and a major concern among ageing men worldwide. The current medical management of PCa typically relies on two key strategies: regular screening and patient triage in risk groups. Regular screening of PCa is performed in men over fifty and includes a digital rectal exam and the measurement of the serum level of the prostate specific antigen (PSA, a PCa biomarker). Risk groups are based on a host of clinicopathological variables, including PSA, the biopsy Gleason score (GS, a histopathological measure of tumor aggressiveness), and the clinical stage (an estimation of tumor size and extent). Then, clinical protocols associate specific treatment options to each risk group (e.g., surgery, radiotherapy, hormonal therapy, and chemotherapy). Thanks to the current clinical management of PCa, prostatic tumors are usually detected and successfully treated at early organ-confined stage, which poses a low to intermediate risk to the patient in about 80% of cases. However, recent population studies suggest that decisions based on the current clinical decision-making protocols may result in some PCa patients being overtreated. Perhaps even more importantly, some patients are estimated to be undertreated. Undertreated patients may experience very rapid growth of aggressive tumors and may eventually succumb to PCa, even after one or several treatments.

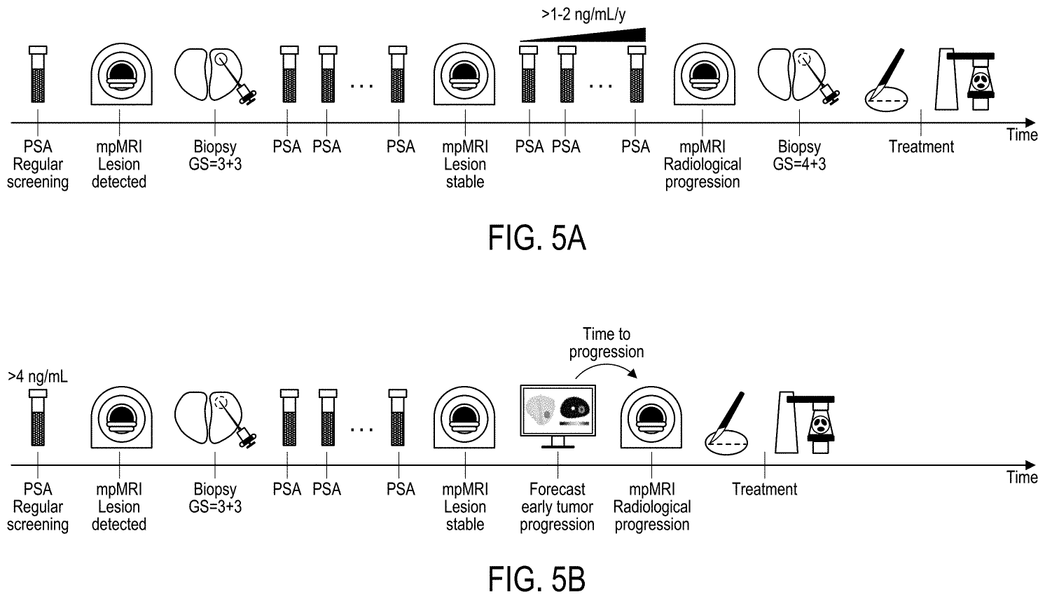

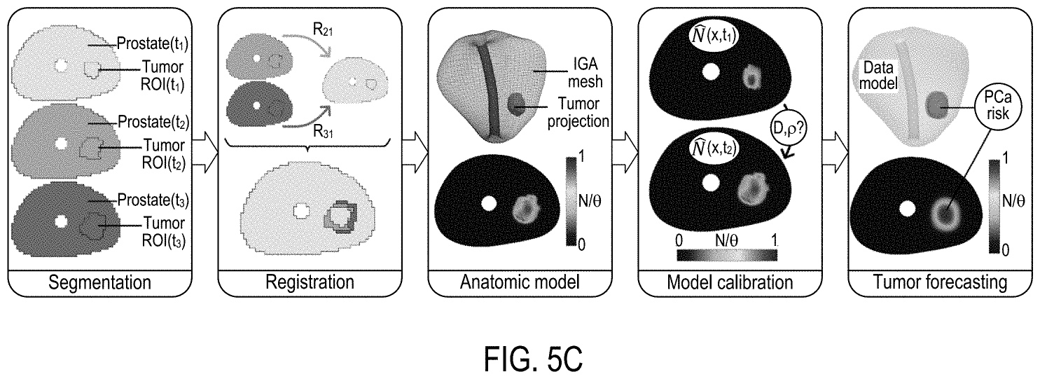

Active surveillance (AS) is a suitable management option for many newly-diagnosed PCa cases, which usually exhibit low to intermediate clinical risk. In AS, patients are monitored via multiparametric magnetic resonance imaging (mpMRI), PSA tests, and biopsies until these reveal an increase in PCa risk that warrants treatment. FIG. 5A depicts a standard-of-care AS protocol for an illustrative patient, according to some embodiments. In the illustrated embodiment, following the detection of a serum PSA level moderately larger than 4 ng/mL, the patient undergoes an initial mpMRI scan that finds an organ-confined cancerous lesion. This radiological lesion is then confirmed as PCa with GS=3+3 in an ensuing biopsy. Since the PCa risk is low, the patient enrolls in AS and periodic PSA tests are performed until the date of the next mpMRI scan in the AS protocol. This second imaging session does not reveal progression in the lesion, so the AS monitoring plan remains unchanged. However, the patient starts exhibiting a fast increase in PSA (e.g., >1-2 ng/mL/year), which motivates an earlier imaging session before the originally prescribed date according to the AS protocol. This third mpMRI scan reveals radiological progression, which is further confirmed histopathologically as an upgrade to GS=4+3 in an ensuing biopsy. At this point, the patient is offered a radical treatment for PCa, which usually consists of surgery (i.e., radical prostatectomy) or radiotherapy (e.g., external beam radiotherapy, brachytherapy).

Accordingly, AS may be implemented to reduce overtreatment of PCa and avoid treatment side-effects that may adversely impact patients' lives without necessarily improving their longevity. Additionally, AS may also enable observation of the dynamics of the patient's tumor, which ultimately contributes to inform therapeutic decision-making and combat undertreatment. Current AS protocols, however, largely rely on assessing PCa according to population-based studies, which typically recommend monitoring tests either at fixed times (e.g., mpMRI every 6-18 months) or after the observation of clinical events suggesting tumor progression (e.g., imaging after a PSA rise, biopsy after worsening of an MM-detected cancerous lesion). This approach complicates the design of personalized plans to ensure a controlled monitoring of a patient's tumor and the early detection of tumor progression. Thus, current challenges in AS include the optimal timing and type of monitoring strategies as well as the triggering criteria and best timing to switch from monitoring to radical treatment (e.g., surgery, radiotherapy).

To address these issues, the present disclosure includes various methods to predict PCa growth during AS using patient-specific forecasts based on an mpMRI-informed biomechanistic model. This model describes the spatiotemporal dynamics of PCa cell density over the patient's prostatic anatomy extracted from T2-weighted (T2 W) MRI data and includes key mechanisms of PCa growth such as tumor cell mobility and proliferation, serum PSA production, and mechanical inhibition due to the forces induced by tumor growth and potentially coexisting benign prostatic hyperplasia (BPH). Tumor cell density may be leveraged since this variable can be estimated from apparent diffusion coefficient (ADC) spatial maps extracted from standard diffusion-weighted (DW) MRI, which enable model initialization, calibration, and validation along with longitudinal PSA values. In many instances, T2 W-MRI and DW-MRI are performed routinely within mpMRI sessions during AS. Accordingly, the disclosed model relies on standard-of-care imaging data. After patient-specific parameterization, personalized model forecasts may enable assessment of the growth of a tumor over the patient's prostatic anatomy in the forthcoming months. In some embodiments, these predictions may be implemented to estimate and forecast the PCa risk using a logistic regression model informed by model-based markers of progression (e.g., mean tumor proliferation, total tumor cell volume). Thus, the PCa forecasts of the present disclosure may let physicians plan the type and timing of the next monitoring tests or curative treatment for each specific patient.

FIG. 5B illustrates the changes to the standard-of-care AS protocol after implementing the computational tumor forecasting pipeline presented in this disclosure, according to some embodiments. The modified protocol is identical to the standard of care up to the second mpMRI. At this point, the longitudinal imaging data collected for the patient can be used to personalize a biomechanistic model of PCa growth and obtain a computational forecast of PCa growth over the patient's prostate anatomy. The forecast reveals progression towards high-risk PCa and provides the time up to this event. This prediction enables to optimize the timing of the third mpMRI to confirm progression and proceed to treatment. Thus, in this example, the disclosed approach avoids PSA testing and biopsy after the second mpMRI, provides a personalized prediction of the patient's PCa progression, and supports the decision and optimal timing to perform treatment. Additionally, PSA testing can be maintained to closely monitor the tumor up to model-predicted date of progression, for example, such that testing frequency is higher if the model predicts faster PSA production due to faster tumor dynamics or PSA tests can be more spaced if the model predicts slow PCa dynamics.

Clinical implementation of the method of the present disclosure may provide a shift from assessing PCa and estimating prognosis based on population-based statistics to actively predicting patient-specific tumor evolution (e.g., PCa forecasting). Actively predicting patient-specific tumor evolution may also present a step towards the optimization of diagnosis, monitoring, and treatment strategies for PCa, hence reducing the current excesses and insufficiencies in PCa treatment.

In various embodiments, preprocessing of patient data may be implemented to begin the active prediction/forecasting techniques disclosed herein. For instance, in certain embodiments, serum PSA data and GS (Gleason score) values from each patient may be stored in a look-up table along with the dates of their assessments. These dates can be further converted to a deidentified timescale, whereby t=0 corresponds, for example, to the date of the first mpMRI scan. Then, the forecasting techniques may include reading these clinical variables along with mpMRI data and their corresponding dates for model calibration and model forecast analysis (e.g., assessment of forecasting performance, construction of a PCa risk classifier using a logistic regression model).

FIG. 5C illustrates the main steps in the disclosed computational pipeline for PCa forecasting during AS, according to some embodiments. In the illustrated embodiment of this study (n=7), all patients had 3 mpMRI scans. Analyzed first is the ability of the disclosed model to recapitulate patient-specific PCa growth after being informed by 3 mpMRI scans. Then, the ability of the model to forecast PCa growth when informed by only the first 2 mpMRI scans is investigated. A third one may then be used to assess the predictive performance of the model. The first step of the computational pipeline is segmentation. The prostate and the tumor region of interest (ROI) may be delineated on the longitudinal mpMRI data collected for the patient. After segmentation, the second and third mpMRI datasets and segments are co-registered with a non-rigid elastic method to the first one (registration transforms R₂₁ and R₃₁, respectively). Next, a virtual model of the prostate anatomy may be built. The model may include a 3D isogeometric mesh and the registered tumor ROIs may be projected over it. Then, the ADC values within each tumor ROI may be mapped to tumor cell density values, which are subsequently used to guide model calibration and find the personalized model parameters. Finally, a patient-specific tumor forecast may be performed, including the prediction of tumor volume, tumor cell density map, and PCa risk.

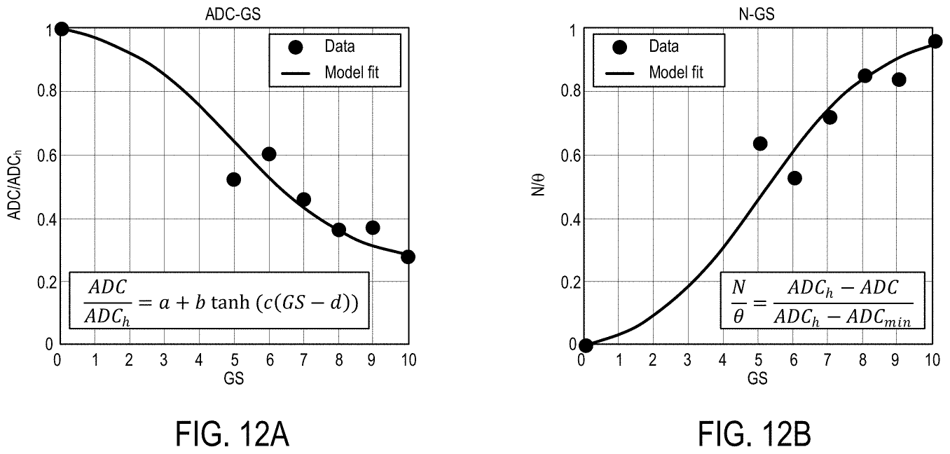

In certain embodiments, the preprocessing of the imaging data has three key steps: segmentation, longitudinal registration, and generation of the patient-specific anatomic model, as shown in FIG. 5C. These steps are briefly described in the following:
Segmentation. The prostate geometry on each of T2 W-MRI series of each of the mpMRI datasets collected for a patient during the course of AS are segmented. This MM series may also be used to segment the central gland of the prostate, which will have different mechanical properties than the peripheral zone (i.e., the rest of the prostate geometry). Additionally, the prostate in the ADC map of each mpMRI dataset may be segmented and the alignment with the corresponding prostate segmentation on the T2 W-MRI series may be checked. In case of misalignment, a rigid or affine registration masking the two MRI imaging series with their corresponding prostate segmentations may be performed. Then, the tumor in each ADC map from each imaging session may be segmented as follows: (i) locate the tumor lesion in both the T2 W images and the ADC map; and (ii) draw a gross tumor region of interest (ROI), such that it includes the observed lesion and a small band of surrounding tissue.
Longitudinal registration. An inter-scan registration of the imaging data and the segmentations may be performed using a nonlinear registration method. This registration can be based on matching the prostate segmentations across the scans using a biomechanical elastic method, or by minimizing the mismatch in the imaging data across the scans. The interscan registration method can further include a penalty term to avoid excessive deformation of the tumor ROI in each imaging dataset. In principle, any of the imaging datasets collected for the patient can be used as the reference dataset for imaging registration, but in practice the typical choice is either the first one or the last one collected for model calibration.
Generation of the patient-specific anatomic model. Once the prostate segmentations have been registered, the one corresponding to the reference imaging dataset may be implemented to build a 3D isogeometric mesh of the patient's prostate using various methodologies. The tumor ROIs may also be projected over the mesh. The tumor ROIs are also used to mask the registered ADC maps. The ADC values of the ADC mask are converted to tumor cell density maps using an ADC-N-GS mapping (shown in FIG. 12). Towards this end, it may be necessary to determine the ADC in healthy tissue (ADCₕ) in each of the imaging scans, which can be obtained as the mean of the ADC values in a region of healthy prostatic tissue far from the tumor with the same volume of the tumor ROI. In each ADC map, the corresponding ADCₕ may be used to normalize the ADC values within the tumor ROI and obtain relative ADC values (i.e., ADC, =ADC/ADCₕ), which can then be mapped to relative tumor cell density values using the ADC-N-GS mapping, as shown in FIG. 12.

In various embodiments, a biomechanistic model may be generated after the preprocessing of the patient data described above. For the biomechanistic model, Ω may represent the patient's prostate and ∂Ω its boundary, which include the external surface of the prostate and the urethra. Then, an embodiment of the biomechanistic model of PCa growth is composed of the following equations:

$$\frac{\partial N}{\partial t} = ∇ \cdot ( {D}_{N} Δ N ) + ρ N ( 1 - \frac{N}{θ} ) \qquad [ 7 ]$$

$$in Ω ; ∇ N \cdot n = 0 in \partial Ω$$

$$\frac{\partial p}{\partial t} = ∇ \cdot ( {D}_{p} ∇ p ) + {α}_{h} ( 1 - \frac{N}{θ} ) + {α}_{c} \frac{N}{θ} - {γ}_{p} p \qquad [ 8 ]$$

$$in Ω ; ∇ p \cdot n = 0 in \partial Ω$$

$${D}_{N} = {D}_{N , 0} {e}^{( - {γ}_{v , D} {σ}_{v} - {γ}_{h , D} \vert {σ}_{h} \vert )} \qquad [ 9 ]$$

$$ρ = {ρ}_{0} {e}^{( - {γ}_{v , ρ} {σ}_{v} - {γ}_{h , ρ} \vert {σ}_{h} \vert )} \qquad [ 10 ]$$

$$∇ \cdot σ = 0 \qquad [ 11 ]$$

$$in Ω ; σ n = - {K}_{w} u in \partial Ω$$

$$σ = λ ( ∇ \cdot u ) I + 2 μ {∇}^{s} u - \frac{K}{3} ( {g}_{N} \frac{N}{θ} + {g}_{BPH} {tH}_{CG} ( x ) ) \qquad [ 12 ]$$

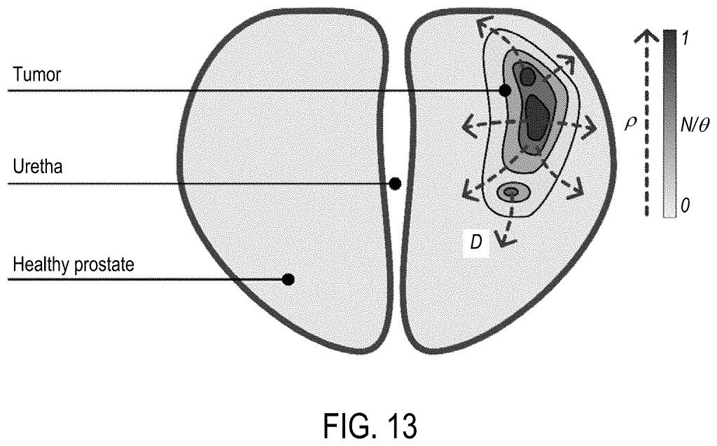

Equation [7] describes the spatiotemporal dynamics of tumor cell density N=N(x,t) as a combination of two mechanisms: (1) tumor cell mobility, which is modelled with a diffusion term (i.e., 1^{st} term in the right-hand side of Equation [7]) and is governed by the tumor cell mobility coefficient (D_{N}); and (ii) tumor cell net proliferation, which is modelled with a logistic reaction term (i.e., 2^{nd} term in the right-hand side of Equation [7]) and is governed by the tumor cell net proliferation rate (ρ). The parameter θ represents the tumor cell carrying capacity, which is the maximum admissible tumor cell density in prostatic tissue. The mechanisms in Equation [7] are illustrated in FIG. 13, described below. This modeling paradigm is common in relation to computational oncology to represent solid tumor growth. In practice, it is common to normalize Equation [7] by dividing both sides of the equation by θ, such that Equation [7] instead models the normalized tumor cell density N/θ, which can therefore only take values in the range [0, 1]. Since the tumors in AS are organ-confined, a no-flux boundary condition may be set.

Equation [8] describes the spatiotemporal dynamics of tissue PSA p=p(x,t), which is the concentration of serum PSA leaked to the bloodstream per unit volume of prostatic tissue. Generally, it may be assumed that the dynamics of tissue PSA are governed by a diffusion process (i.e., 1^{st} term in the right-hand side of Equation [8]) that is controlled by diffusivity Dₚ, and three reaction terms (i.e., three last terms in the right-hand side of Equation [8]). These reaction terms represent the production of tissue PSA in healthy tissue at rate αₕ, the production of tissue PSA in tumor tissue at rate α_{c}, and a natural decay rate γₚ, respectively. It may be assumed that tissue PSA cannot flow outside the prostate boundary, so a no-flux boundary condition may be set. To calculate the serum PSA (Pₛ) used in the clinic, it may suffice to integrate the tissue PSA field over the patient's prostate domain:

Pₛ(t)=∫_{Ω}p(x,t)dΩ [13]

Equations [9] and [10] describe the inhibition of PCa caused by the local mechanical stress σ=σ(x,t) induced by the growing tumor and coexisting BPH. The parameters governing tumor dynamics in Equation [7] may be modified using an exponentially decaying mechanotransductive function that depends on two scalar metrics of the local mechanical stress field: (i) the von Mises stress σᵥ, which accounts for tissue distortion, and (ii) the hydrostatic stress σₕ, which accounts for the natural accumulation of stress within the growing tumor. Hence, in Equations [9] and [10], D_{N,0} and ρ₀ are the baseline values of the tumor cell mobility coefficient and the tumor cell net proliferation rate, respectively. Additionally, the parameters γ_{v,D}, γ_{h,D}, γ_{v,ρ}, and γ_{h,ρ} are mechanotransductive constants to couple the mechanical state in the prostate with tumor dynamics. Since both tensile and compressive hydrostatic stress in in vivo scenarios can inhibit solid tumor growth, the absolute value of this mechanical stress metric may be taken. The formulation of Equations [9] and [10] may be based on previous studies of mechanical inhibition of solid tumor growth.

In Equation [11], the deformation of the prostate under PCa growth and BPH may be assumed to be a quasistatic phenomenon. This is a common assumption for computational modeling of tumor growth. The mechanical boundary conditions of the prostate may be modelled using a Winkler-inspired formulation. These boundary conditions aim at representing the confinement of the prostate in the pelvic region and, hence, the reaction of surrounding tissues and organs to prostate volumetric growth resulting from PCa growth and BPH. Thus, these boundary conditions depend on the displacement vector u=u(x,t) evaluated on the prostate boundary ∂Ω, and a rigidity matrix K_{w}=K_{w}(x,t), which may vary over the prostate boundary and during the pathological deformation of this organ. Nevertheless, in practice, it can take a simplified form K_{w}=k_{w}I, where I is the 3×3 identity matrix and k_{w} represents the same rigidity along the three directions of space.

In Equation [12], the prostatic tissue may be modelled as a linear elastic, heterogeneous, isotropic material. In Equation [12], λ and μ are the Lame parameters, which are calculated from the Young modulus E and the Poisson ratio v. A high value for the Poisson ratio may be adopted to represent the high water content of the prostatic tissue, which is a common choice for other solid tumor growth models. As the central gland of the prostate is typically stiffer than the peripheral zone, a larger Young modulus may be adopted in the central gland. The Young modulus over the prostate can thus be assumed to be a spatial function E=E(x), which takes different values in the peripheral zone and the central gland. These values can be constants in either the anatomic region of the prostate, which is a usual practical choice, or can be spatial maps inferred from imaging data (e.g., mpMRI to identify stiffer zones due to BPH nodules or PCa). The last term in the right-hand side of Equation [12] represents the mechanical stresses introduced by the growth processes in the prostate: PCa and coexisting BPH, respectively. The parameter K is the bulk modulus, which is calculated from the local values of the Young modulus and Poisson ratio. For PCa, a term that linearly depends on the normalized tumor cell density through a rate g_{N} may be used. For BPH, a linear term that is only defined on the central gland may be used thanks to the Heaviside function H_{CG}(x), and that depends on a rate g_{BPH} and time t.

In some embodiments, Equation [7] may also be coupled with another equation representing nutrient dynamics. The nutrient field may then inform the tumor cell mobility coefficient D_{N}, the tumor cell proliferation rate ρ, and/or the carrying capacity θ. For example, this can be done using various known formulations proposed for a vascular density field. In some embodiments, other hyperelastic constitutive models may be proposed to replace the linear elastic model in Equation [12]. Under a hyperelastic constitutive model, the mechanical properties of the prostatic and tumor tissue can vary over space and time due to the deformation process.

In various embodiments, numerical methods, such as those described herein, may be implemented to solve the above-described biomechanistic model. For instance, in some embodiments, isogeometric analysis may be used to discretize in space based on a 3D, quadratic, C¹ Non-Uniform Rational B-spline (NURBS) functional basis. This functional basis can be used to build an anatomic model of the patient's prostate. The generalized-alpha method may be used to integrate in time. Additionally, a staggered scheme, where the mechanical equilibrium is solved every 2-5 time steps, may be used while tumor and tissue PSA dynamics are solved in all time steps (assuming a time step of approximately Δt=l·day).

In certain embodiments, the biomechanistic model is initialized by first extracting the tumor cell density from the first mpMRI collected from a patient where PCa is visible as the initial conditions for Equation [7]. Second, the serum PSA value collected at the date of the first mpMRI date (or its estimate at this date using the other serum PSA values collected at other dates for the patient) is used. Finally, the mechanical stress generated by BPH in the prostate up to the first mpMRI date is estimated. In some embodiments, an inverse problem is run using Equations [11] and [12], such that Equation [12] is only equipped with the BPH term. This inverse problem may find the BPH growth rate (g_{BPH}) and constant Winkler constants (k_{w}) that best match the BPH deformation of the patient's prostate from an atlas-based prostatic geometry (assuming this was the healthy state at age 40) up to the patient's prostate geometry at the date of the first mpMRI scan. The mechanical stress induced by BPH before the first scan is a byproduct of this calculation. Then, to estimate the mechanical stresses generated by PCa at the onset of model simulations, the mechanical equilibrium at the date of the first mpMRI scan may be solved using Equations [11] and [12], such that Equation [12] includes the PCa term initialized with the tumor cell density from the first mpMRI scan.

After the model is initialized, various methods may be used to calibrate the model. Standard practice involves using nonlinear least-squares methods (e.g., gradient descent, Gauss-Newton, Levenberg-Marquardt) or adjoint methods. These methodologies rely on the minimization of an objective functional J, which may be defined as follows:

$$J = {w}_{1} {\sum }_{i} \frac{{\int }_{Ω} {( N ( x , {t}_{i} ) - \hat{N} ( x , {t}_{i} ) )}^{2} d Ω}{{\int }_{Ω} {( \hat{N} ( x , {t}_{i} ) )}^{2} d Ω} + {w}_{2} {\sum }_{j} \frac{{( {P}_{s} ( {t}_{j} ) - \hat{{P}_{s}} ( {t}_{j} ) )}^{2}}{{( \hat{{P}_{s}} ( {t}_{j} ) )}^{2}} + {w}_{3} {\sum }_{k} \frac{{( {p}_{k} - {p}_{k , ref} )}^{2}}{{p}_{k , ref}^{2}} \qquad [ 14 ]$$

The first term in the right-hand side of Equation [14] accounts for the mismatch between the model prediction and the imaging measurement of the tumor cell density maps at each of the imaging scans available for model calibration (i=1, . . . , n_{MRI}). Likewise, the second term in the right-hand side of Equation [14] accounts for the mismatch in the serum PSA predicted by the model and measured in the patient at each of the dates of PSA testing after the first mpMRI scan (j=1, . . . , n_{PSA}). The last term in the right-hand side of Equation [14] is a regularization term, which can be activated for all or some of the pₖ (k=1, . . . , nₚ) parameters to be calibrated. In this term, p_{k,ref} is a reference value for the parameter (e.g., the mean value obtained for a population of patients) around which to find the patient-specific value. Finally, the weights w₁, w₂, and w₃ enable adjustment of the relative contribution of each of the three terms. For example, this can be convenient to facilitate model calibration in the case of excessive noise in the imaging data or large oscillations of PSA during AS.

Depending on the availability of imaging and PSA data for each patient, some of the parameters in the model in Equations [7]-[12] can be fixed to constant value from the literature or previous population studies. These parameters include: (i) the carrying capacity θ, which can be fixed according to prostate tissue cell size and packing or can be eliminated through normalization of the tumor cell density; (ii) the diffusivity of the tissue PSA Dₚ, which can be assumed to be similar to a tumor cell mobility coefficient D_{N}; (iii) the production rate of tissue PSA in healthy tissue, which can be estimated from the literature based on prostate size; (iv) the tissue PSA decay rate γₚ, which may be estimated to yield a serum PSA half-life of 2 to 3 days according to large studies of post-surgery PSA changes in patients undergoing prostatectomy; (v) the mechanotransductive coupling constants γ_{v,D}, γ_{h,D}, γ_{v,ρ}, and γ_{h,ρ}, the mechanical properties of the tissue (e.g., the Young modulus of the prostate tissue regions, the Poisson ration, and the Winkler constant k_{w}) may be fixed; and (vi) the deformation rates of PCa and BPH (i.e., g_{N} and g_{BPH}, respectively) may also be fixed during the simulation.

In some embodiments, the minimal set of parameters that require patient-specific calibration may be assumed to be the baseline value of the tumor cell mobility and tumor cell proliferation rate (D_{N,0} and ρ₀, respectively), along with the tissue PSA production rate (α_{c}). Hence, the minimal amount of data to calibrate the model using the method outlined above may include two mpMRI scans and two PSA measurements.

In various embodiments, the outcome of model calibration described above is a personalized set of parameters, with which personalized forecasts of PCa growth may be run. These forecasts may inform the physician about key features of PCa, such as the rate of PSA increase, the increase in estimated GS, the tumor volume, and the probability of extracapsular expansion (e.g., if a large part of the tumor volume has reached the prostate boundary). Accordingly, based on the model forecasts, the physician could decide the frequency of upcoming PSA tests, mpMRI scans, and biopsies as well as whether the patient requires immediate treatment or may continue in AS.

In some embodiments, parameter distributions may be sampled for a specific patient at the time of model initialization, which includes one mpMRI scan and a single value of the serum PSA at the same date. Running a model simulation for each sample would produce a distribution of model outcomes of interest (e.g., serum PSA, GS, tumor volume) that could be used to estimate the probability of tumor progression in several time horizons (e.g., 1 year, 2 years, 3 years, etc.). These results can be used to guide clinical decision making as outlined above (e.g., frequency of monitoring tests or treatment versus AS).

In certain embodiments, a logistic regression model is trained to provide a classifier of low-risk PCa (e.g., GS=3+3 or lower) versus high-risk PCa (e.g., GS=3+4 or higher). This logistic regression model may be trained upon the personalized biomechanistic model informed by all the data from each of the patients in a cohort (e.g., mpMRI scans, serum PSA values, and GS measurements in biopsy and surgery). Towards this end, the spatiotemporal maps of tumor cell density N(x,t) and tissue PSA p(x,t) obtained from each patient-specific model simulation over the course of AS can be integrated over the patient's prostate to render time-resolved trajectories of model-based metrics of interest to feed the logistic regression model. These model-based markers include:
The prostate volume V_{P}=∫_{Ω}d_{Ω}
The tumor volume V_{T}=∫_{Ω}_{_{T}}d_{Ω}, where Ω_{T} is the tumor region defined as the region of space where N>Nₜₕ and Nₜₕ is the threshold value to identify tumor tissue according to the biomechanistic model.
The total tumor cell volume V_{N}=∫_{Ω}_{_{T}} N(x,t)/θ dΩ
The mean normalized tumor cell density

$$\overline{N} / θ = ( {\int }_{{Ω}_{T}} N ( x , t ) / θ d Ω ) / ( {\int }_{{Ω}_{T}} dΩ = \frac{{V}_{N}}{{V}_{T}} )$$

The total tumor index N_{T}=N̅V_{T}/V_{P}
The mean proliferation activity of the tumor

$${P}_{m} = {\int }_{{Ω}_{T}} ρ N ( x , t ) ( 1 - \frac{N ( x , t )}{θ} ) d Ω$$

The serum PSA Pₛ=∫_{Ω}p(x,t)dΩ
The serum PSA velocity

$${v}_{{P}_{s}} = {\int }_{Ω} \frac{\partial p}{\partial t} ( x , t ) d Ω .$$

Once these metrics are calculated over the time that each patient has been under AS, a hierarchy of univariate and multivariate logistic regression models may be built using one or multiple metrics from the list above to select the best one according to classifier performance under receiver operator characteristic (ROC) curve analysis and model complexity (e.g., minimal amount of biomechanistic model-based metrics). Usual metrics of classifier performance in ROC curve analysis include area under the ROC curve (AUC) as well as sensitivity, specificity, and accuracy at the optimal performance point (e.g., calculated using the Youden index or the minimum distance of the ROC curve points to the upper left corner of the ROC curve plot).

Once the classifier is constructed, it may be used to forecast PCa along with tumor cell density and serum PSA in patients in AS following the calibration of the biomechanistic model. To this end, the trained logistic regression model may be fed with the personalized forecasts of the trajectories of the chosen biomechanistic model-based markers that rendered the best classifier performance.

In certain embodiments, the capability to predict PCa risk is key to guide decision-making in AS since it provides a solid basis to early identify patients that will exhibit tumor progression towards higher-risk disease, decide the frequency of monitoring tests to confirm this progression based on the model predicted time to progression, and decide whether the patient can stay in AS or requires immediate treatment. In other words, the disclosed methodology to predict PCa risk for each patient exploits the personalized biomechanistic model forecasts to guide the personalized design of AS plans and provide a robust criterion to trigger radical treatment for each individual patient, which are two coveted demands in current AS of PCa.

### Examples Related to MRI-Based Forecasting

A preliminary study of the above-described forecasting techniques was conducted using only mpMRI and GS values, and considering only PCa dynamics (e.g., only Equation [7] in the system consisting of Equations [7]-[12], and only using the first right-hand side term in the objective functional for model calibration in Equation [14]). This preliminary study was conducted in a cohort of seven PCa patients who enrolled in AS and had three mpMRI scans over a period of 1.4 to 4.9 years. All patients had imaging and histopathologically-confirmed PCa via biopsy and/or analysis of the surgically-removed prostate after AS. The patients in the cohort had biopsies before and after the first mpMRI scan, which was set as the beginning of the relative time scale for each patient (t=0). All the biopsy GS measurements after the first mpMRI scan were considered eligible for this study. The biopsy GS data that were collected more than one year before the first mpMRI were eliminated if there was another GS measurement collected in the three months following the first mpMRI; otherwise, if there were two GS measurements with the same value before and after the first mpMRI and such that the first post-imaging value is obtained more than 3 months after the mpMRI scan, then we set that GS value at t=0. No patient had a single GS measurement collected more than one year before the first mpMRI. This selection of eligible GS measurements resulted in 16 eligible values distributed in 8 being 3+3, which were identified as lower-risk PCa, and another 8 being larger or equal than 3+4, which were classified as higher-risk PCa. This dichotomy is introduced on the basis if GS 3+3 being extensively recognized as eligible for AS, while there is debate regarding the inclusion in AS protocols of PCa cases exhibiting Gleason grade 4.

Three analyses were conducted in the preliminary study: (i) global calibration to recapitulate PCa growth in each patient using the simplified biomechanistic model as outlined above, (ii) fitting-forecasting to analyze the prediction of PCa growth with the simplified biomechanistic model, and (iii) PCa risk identification using a logistic regression model.

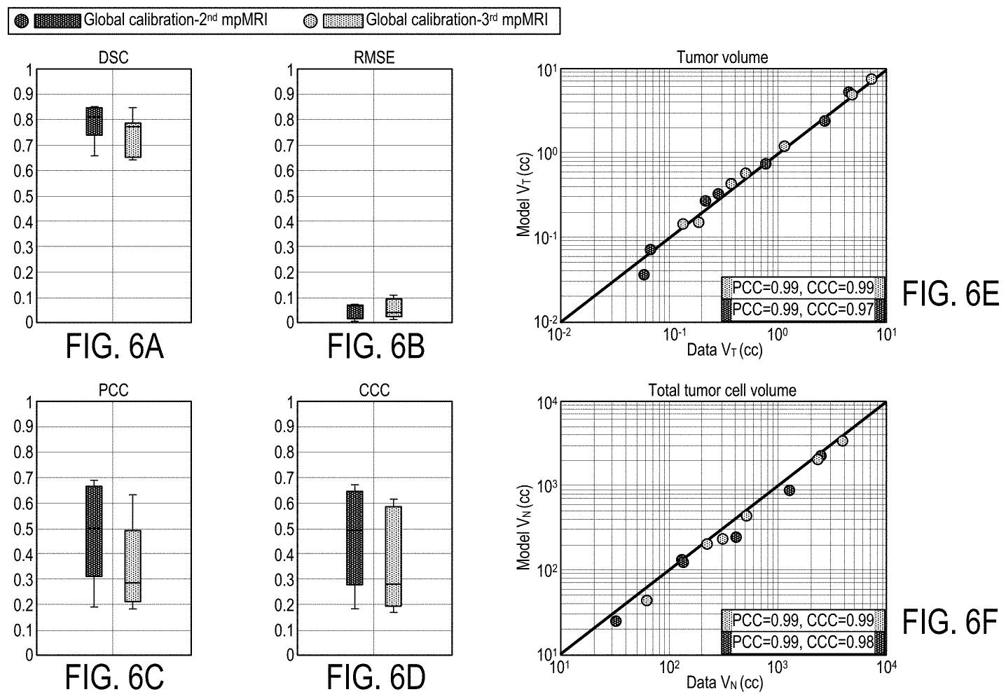

Global calibration. In this study, the simplified biomechanistic model is informed with the 3 mpMRI datasets collected for each patient. Hence, the first one is used to initialize the model and the other two are used to calibrate the model and find the values of the two parameters governing PCa dynamics in the simplified biomechanistic model (e.g., the tumor cell mobility D_{N} and the tumor cell net proliferation p). FIGS. 6A-F summarize the model performance to recapitulate PCa growth in AS, according to some embodiments. FIGS. 6A-D show the distributions of four metrics assessing the agreement between the tumor cell density map calculated with the biomechanistic model and extracted from mpMRI data across the patient cohort (n=7) in the global calibration scenario: (A) Dice Similarity Coefficient, (B) Root Mean Squared Error, (C) Pearson Correlation Coefficient, and (D) Concordance Correlation Coefficient. These metrics were calculated at the dates of the 2^{nd} and 3^{rd} mpMRI scans (blue and green boxplots, respectively). FIGS. 6E and 6F show unity plots to assess the agreement between the model estimation and mpMRI measurement of the tumor volume and the total tumor cell volume across the patient cohort (n=7), respectively. These tumor quantities were calculated at the dates of the 2^{nd} and 3^{rd} mpMRI scans (blue and green points, respectively). The unity plots in FIGS. 6E and 6F further report the corresponding Pearson (PCC) and Concordance Correlation Coefficients (CCC) for the tumor quantities at the dates of the 2^{nd} and 3^{rd} mpMRI scans.

A remarkable model-data agreement was obtained in tumor volume and total tumor cell volume in terms of the Pearson and concordance correlation coefficient (PCC and CCC). Additionally, at the local level an overall good agreement was observed in the tumor cell density maps obtained with the model at the dates of the 2^{nd} and 3^{rd} mpMRI scans and the corresponding tumor cell density maps obtained from the ADC maps in these imaging sessions. This local agreement was measured in terms of the Dice Similarity Coefficient (DSC), root mean-squared error of the normalized tumor cell density (RMSE), and local PCC and CCC. There were no significant differences found between these local metrics calculated at the date of the second versus the third mpMRI date under a two-sided Wilcoxon rank-sum test (p>0.05). However, the differences were significant under a two-sided Wilcoxon sign-rank test (p<0.05). This result suggests that the biomechanistic model may perform better in recapitulating PCa growth in the short term after model initialization than in the long term. To achieve better performance in the long-term, the model may need to include further spatiotemporally-resolved mechanisms to inform PCa dynamics, such as the mechanical inhibition proposed in Equations [9-12] or a nutrient availability map as suggested herein.

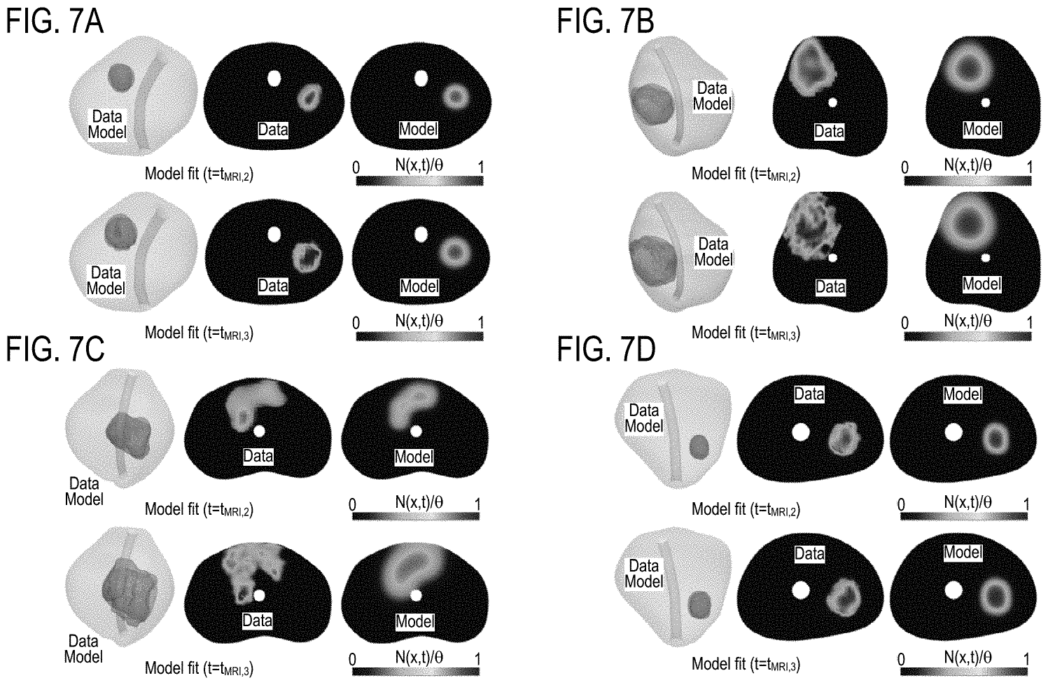

Additionally, FIGS. 7A-D illustrate the model outcomes for four patients, including the tumor geometry within the 3D anatomic model of each patient's prostate and an axial section of the prostate showing the normalized tumor cell density map obtained with the model and from the ADC measurements at the second and third mpMRI sessions. FIGS. 7A-D illustrate the recapitulation of PCa growth at the dates of the 2^{nd} mpMRI scan during model calibration (upper row in each panel) and the 3^{rd} mpMRI scan (bottom row in each panel) in four patients, respectively.

FIGS. 7A-D show that, while the simplified model can reasonably capture the tumor volume and total tumor cell volume, further research may be necessary to achieve a higher spatial agreement between the tumor cell density maps obtained with the model from ADC maps. Achieving this improvement in the model performance would also contribute to increase the DSC as well as the local PCC and CCC, while also reducing the RMSE.

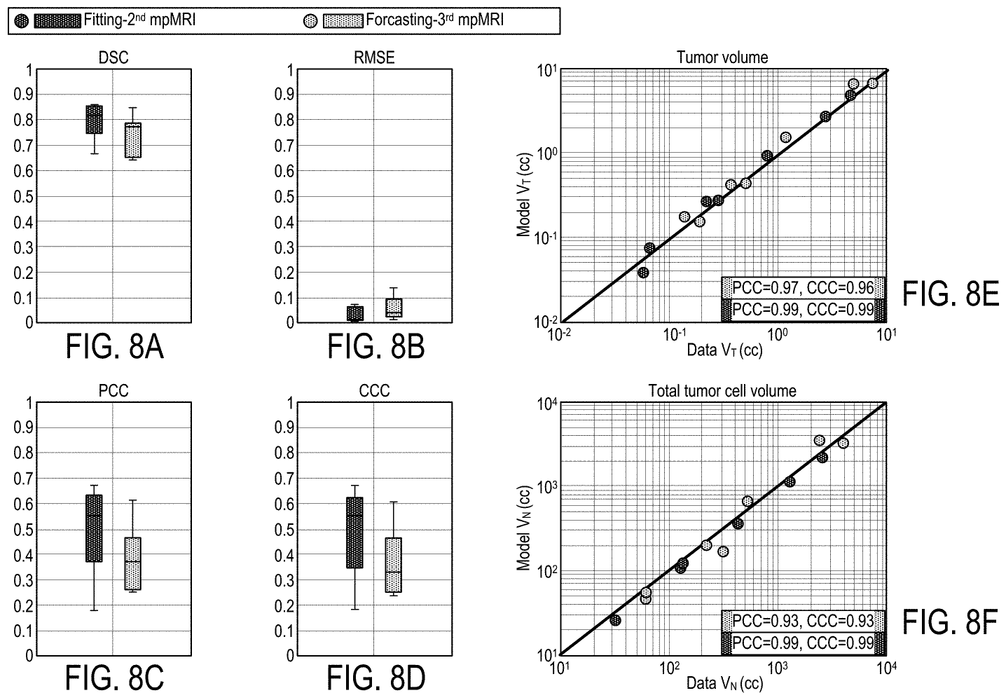

Fitting-Forecasting. In this study, the biomechanistic model is informed with only the first two mpMRI datasets collected for each patient and the third one is used for validation of the model forecasts. Hence, the first imaging dataset is used to initialize the model, while the second is used to calibrate the model and find the values of the two parameters governing PCa dynamics in the simplified biomechanistic model (e.g., the tumor cell mobility D_{N} and the tumor cell net proliferation ρ). FIGS. 8A-F summarize the model performance to recapitulate PCa growth between the first and second mpMRI, as well as the model performance in forecasting PCa growth at the date of the third mpMRI. FIGS. 8A-F show the distributions of four metrics assessing the agreement between the tumor cell density map calculated with the biomechanistic model and extracted from mpMRI data across the patient cohort (n=7) in the fitting-forecasting scenario: (FIG. 8A) DSC, (FIG. 8B) RMSE, (FIG. 8C) PCC, and (FIG. 8D) CCC. These metrics were calculated at the dates of the 2^{nd} and 3^{rd} mpMRI scans (blue and green boxplots, respectively). FIGS. 8E and 8F show unity plots to assess the agreement between the model estimation and mpMRI measurement of the tumor volume and the total tumor cell volume across the patient cohort (n=7), respectively. These tumor quantities were calculated at the dates of the 2^{nd} and 3^{rd} mpMRI scans (blue and green points, respectively). The unity plots in FIGS. 8E and 8F further report the corresponding Pearson (PCC) and Concordance Correlation Coefficients (CCC) for the tumor quantities at the dates of the 2^{nd} and 3^{rd} mpMRI scans.

As shown in FIGS. 8A-8F, a remarkable model-data agreement is obtained in tumor volume and total tumor cell volume in terms of PCC and CCC both at the calibration endpoint (e.g., at the date of the second mpMRI) at the forecasting horizon (e.g., the date of the third mpMRI scan). Furthermore, again an overall good agreement is observed at local level in term of the DSC, RMSE, local PCC, and local CCC assessing the model-data mismatch in the tumor cell density map at the calibration endpoint and forecasting horizon (2^{nd} and 3^{rd} mpMRI scan dates, respectively). As in the global calibration scenario, no significant differences were found between these local metrics calculated at the date of the second versus the third mpMRI date under a two-sided Wilcoxon rank-sum test (p>0.05). However, the differences were significant under a two-sided Wilcoxon sign-rank test (p<0.05), thereby suggesting to extend the model with spatiotemporally-resolved mechanisms as mentioned above. Additionally, neither two-sided Wilcoxon rank-sum tests nor two-sided Wilcoxon signed-rank tests found significant differences (p>0.05) in the parameter values, global metrics, or local metrics calculated either at the date of the 2^{nd} mpMRI or the date of the 3^{rd} mpMRI in the global calibration versus the fitting-forecasting scenario. Thus, this result suggests that the biomechanistic model may exhibit a forecasting performance between the 2^{nd} and 3^{rd} mpMRI scans that is non-inferior to the recapitulation of PCa dynamics in the same timeframe if the model had been informed with the 3^{rd} mpMRI scan.

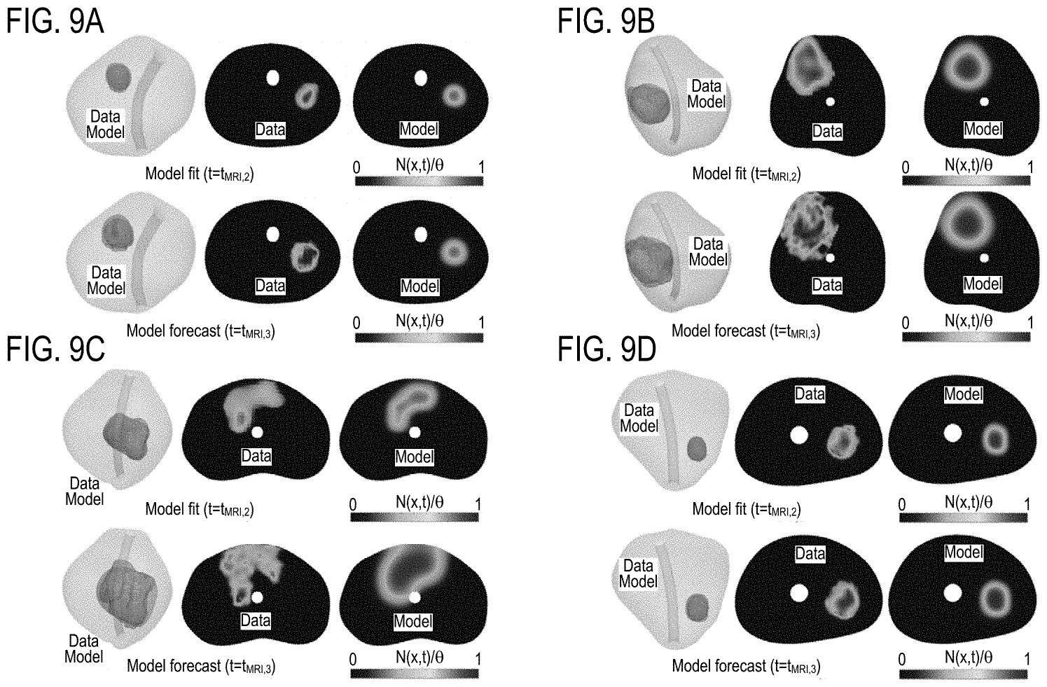

FIGS. 9A-9D further illustrates the model forecasts for the same four patients in FIGS. 7A-7D, including the tumor geometry within the 3D anatomic model of each patient's prostate and an axial section of the prostate showing the normalized tumor cell density map obtained with the model and from the ADC measurements at the calibration endpoint (2^{nd} mpMRI) and forecasting horizon (3^{rd} mpMRI). FIGS. 9A-9D illustrate the recapitulation of PCa growth at the date of the 2^{nd} mpMRI scan during model calibration (upper row in each panel) and the ensuing prediction of tumor growth at the date of the 3^{rd} mpMRI scan (bottom row in each panel) in four patients, respectively.

FIGS. 9A-9D show again that, while the simplified model can reasonably capture the tumor volume and total tumor cell volume, further research may be necessary to achieve a higher spatial agreement between the tumor cell density maps obtained with the model from ADC maps. Achieving this improvement in the model performance would also contribute to increase the DSC as well as the local PCC and CCC, while also reducing the RMSE.

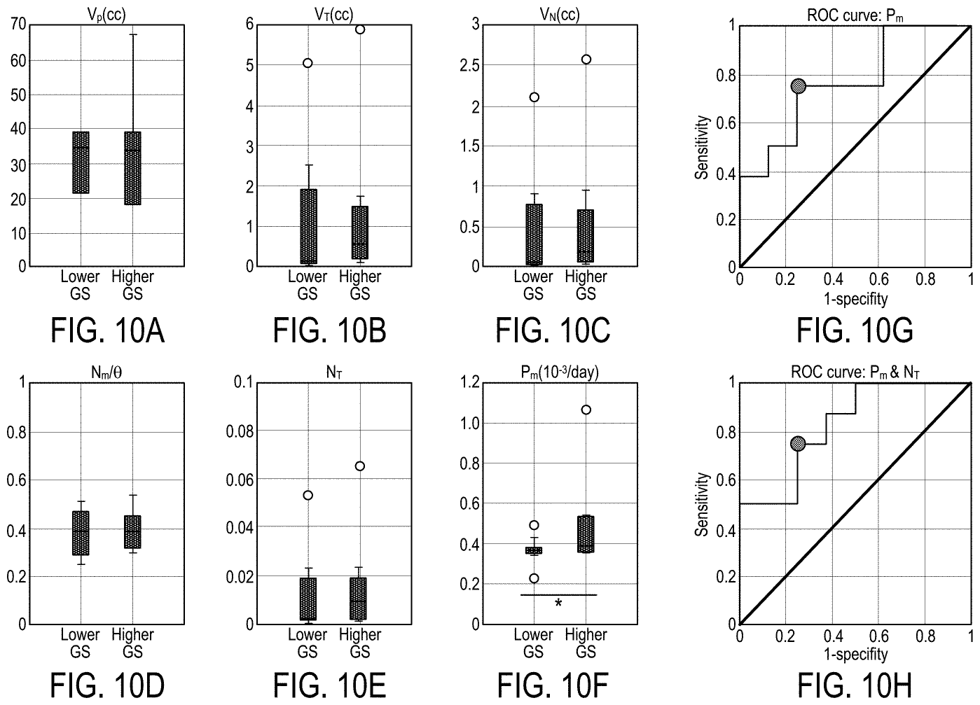

Logistic regression model to build a PCa risk classifier. The personalized model simulations from the global fitting scenario were used to calculate a panel of six potential model-based biomarkers of high-risk PCa (e.g., GS larger or equal to 3+4) at the times of histopathological assessment of the patients' tumor using biopsy or after radical prostatectomy (n=16). FIGS. 10A-10H show the resulting distribution of these six potential model-based biomarkers in lower-risk (n=8) and higher-risk (n=8) PCa, which were defined as tumors with GS=3+3 and GS≥3+4, respectively. These model-based markers are: (FIG. 10A) prostate volume, (FIG. 10B) tumor volume, (FIG. 10C) total tumor cell volume, (FIG. 10D) mean normalized tumor cell density, (FIG. 10E) total tumor index, and (FIG. 10F) mean proliferation activity of the tumor. Outliers are represented as hollow circles and an asterisk indicates significance under a one-sided Wilcoxon rank-sum test (p<0.05).

As shown in FIGS. 10A-10F, only the mean proliferation activity of the tumor was significantly larger in higher-risk GS under a one-sided Wilcoxon rank-sum test (p=0.041). Higher proliferation rates in tumors with higher GS have been shown in past studies. Additionally, a non-significant trend is observed towards higher tumor volume, higher total tumor cell volume, and higher total tumor index in higher-risk PCa cases. These results are also supported by observations of a positive correlation between higher tumor volume and higher cellularity with higher GS.

A univariate logistic regression model was then constructed to classify lower-risk PCa versus high-risk PCa using the values of the mean proliferation activity of the tumor at the times of histopathological assessment. The ROC curve for this univariate classifier is shown in FIG. 10G, achieving an AUC=0.77 and operating at 75% sensitivity and specificity at optimal point. Five bivariate classifiers resulting from building logistic regression models were considered by using the mean proliferation activity of the tumor combined with only one of the other five potential model-based biomarkers. FIG. 10H shows the ROC curve of the best performing bivariate classifier, which leveraged the total tumor index. The resulting AUC in 0.83 and the bivariate classifier also performs at 75% sensitivity and specificity at optimal point. Given the reduced size of the cohort, classifiers involving more than two model-based biomarkers were not considered.

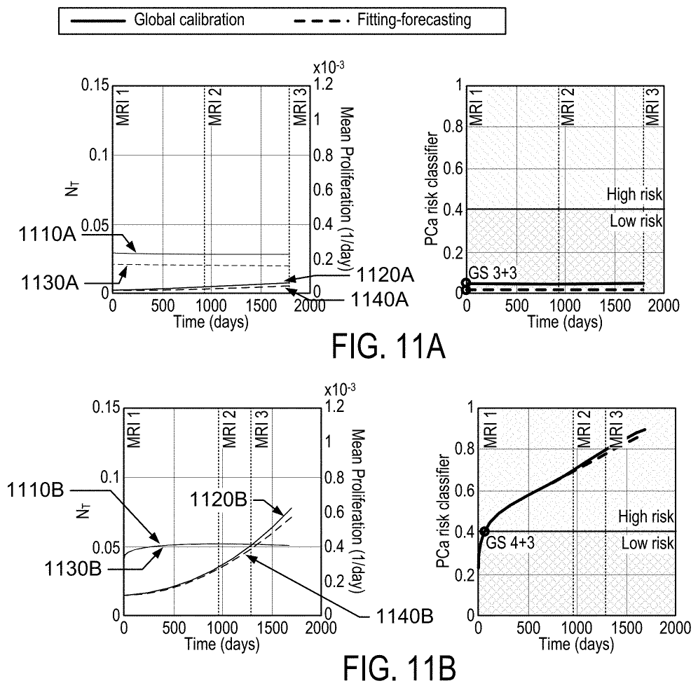

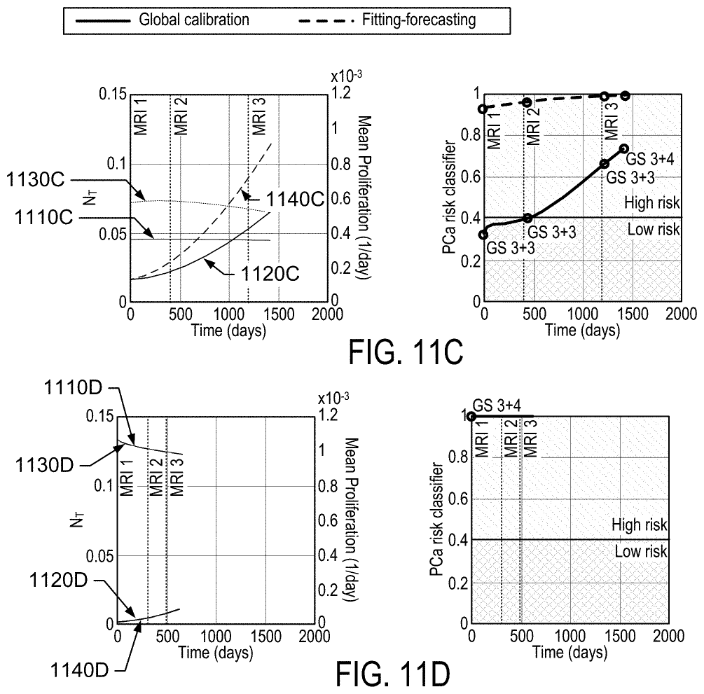

Finally, the trajectory of the bivariate classifier (as trained in the global calibration scenario) was analyzed over the course of AS using the biomechanistic model results from both the global calibration scenario and the fitting-forecasting scenario. FIGS. 11A-11D show the time trajectories of the model-based markers involved in the calculation of the PCa risk classifier (left) as well as the trajectory of the latter (right) for four patients, respectively. These patients are the same as the ones reported in FIGS. 7A-7D and 9A-9D. Lines 1110A-D correspond to results calculated from the global calibration scenario (see FIGS. 7A-7D) with the model-based markers of interest being the mean proliferation activity of the tumor (right vertical axis). Lines 1120A-D correspond to results calculated from the global calibration scenario (see FIGS. 7A-7D) with the model-based markers of interest being the total tumor index N_{T} (left vertical axis), which is calculated as the product of the mean tumor cell density of the tumor and the ratio of the tumor volume to the prostate volume. Lines 1130A-D correspond to results obtained from the fitting-forecasting scenario (see FIGS. 9A-9D) with the model-based markers of interest being the mean proliferation activity of the tumor (right vertical axis). Lines 1140A-D correspond to results obtained from the fitting-forecasting scenario (see FIGS. 9A-9D) with the model-based markers of interest being the total tumor index N_{T} (left vertical axis). Dotted vertical lines in the background indicate the times of the mpMRI scans for each patient.

The PCa risk classifier was trained with the global calibration results, yielding an optimal performance threshold that separates lower risk PCa (lower region) from higher-risk PCa (upper region). The PCa risk at the times of histopathological assessment of the patients' tumors (i.e., biopsy, examination of the surgical specimen) is represented as a hollow circle, and the corresponding GS values are annotated. In FIG. 11A, the patient exhibits a low-risk tumor during AS, which is correctly represented by our model in both computational scenarios. In FIG. 11B (note that lines 1110B and 1130B closely overlap in the figure), the model consistently classifies the patient's tumor as higher-risk during the majority of AS in both computational scenarios. In FIG. 11C (note that lines 1110C and 1120C cross in the figure), the patient initially exhibits a lower-risk tumor that had progressed to higher-risk at the time of surgery (i.e., terminal GS value). In the fitting-forecasting scenario (lines 1130C, 1140C), the model consistently predicts that the tumor is high risk, while in the global calibration scenario (liens 1110C, 1120C), tumor progression is detected shortly after the second mpMRI scan. Advantageously, in the fitting forecasting scenario, the disclosed approach identifies a high-risk tumor 1011 days earlier than standard practice (i.e., assessment of the surgical specimen). In FIG. 11D (note that lines 1110D and 1130D closely overlap and lines 1120D and 1140D closely overlap in the figure), the disclosed model consistently identifies the tumor as high risk in both computational scenarios.

Additionally, using the global fitting results, the trajectory of the bivariate PCa risk classifier correctly identified all non-progressing PCa (2/7 patients) and progressing PCa (5/7 patients) towards higher-risk disease in the cohort. In the fitting-forecasting scenario, it was observed that there is a trend towards overestimating the mean proliferation activity of the tumor (especially in the higher-risk tumors), which would render conservative estimations of PCa risk (e.g., on the side of more secure predictions). Nevertheless, the bivariate classifier correctly identified the five progressing tumors and one of the non-progressing tumors. Of note, this classification would be performed at the date of the second mpMRI scan which can dramatically anticipate the detection of progressing disease and reduce undertreatment of PCa. For example, the patient in FIG. 11D, as described above, would be diagnosed with progressing PCa 1011 days (˜2.8 years) earlier than standard practice (in this case, assessment of the surgical specimen at the end of the trajectory).

Although not all tumors were correctly classified as lower-risk or higher-risk at the date of histopathological assessment (recall that the classifier performs at 75% sensitivity and specificity in the ROC curve analysis) in the above-described examples, the trajectory of the bivariate classifier exhibits a promising performance in identifying progressing PCa in both the fitting-forecasting and global calibration scenarios. Advantageously, this trajectory may be calculated on a patient-specific basis by informing the bivariate classifier with the trajectories of biomarkers calculated from the personalized simulations of PCa growth obtained from the biomechanistic model. Thus, the combination of the personalized forecast of spatiotemporal PCa growth along with a personalized prediction of PCa risk may provide a promising approach to inform treating physicians in clinical decision-making for AS in two crucial aspects. First, these forecasts can inform the frequency of monitoring tests (e.g., PSA tests, mpMRI scans, biopsy), for example, prescribing more sparse and less invasive tests (e.g., sparse PSA tests and an mpMRI in a 1-2 year horizon) if the tumor shows slow dynamics and lower risk for the considered forecasting window versus more frequent and invasive tests (e.g., periodic PSA tests, an mpMRI within 6-12 months, and/or an ensuing biopsy after imaging) if the tumor exhibits fast dynamics or is predicted to progress during the forecasting window. This capability may allow the design of personalized AS protocols based on the results of the personalized forecasts of PCa risk and PCa growth over the patient's prostate. Additionally, refining the model formulation to yield an improved spatial model-data agreement for tumor cell density maps may enable the calculation of accurate local GS maps obtained from the tumor cell density maps by applying the ADC-N-GS mapping.

These local GS maps could then be instrumental to guide biopsy to the areas of the tumor predicted to exhibit larger GS values. Second, the spatiotemporal PCa growth and PCa risk forecasts may be leveraged to define robust criteria to trigger treatment in patients predicted to undergo tumor progression. Therefore, the PCa forecasting technology described herein may be implemented in AS to advantageously shift from a population-based, observational standard to a patient-specific, predictive strategy.

FIGS. 12A and 12B illustrate ADC-N-GS mapping, according to some embodiments. FIG. 12A shows a nonlinear least-squares fit of a hyperbolic tangent model (solid line) to the mean values of ADC across GS groups in previous studies (dots), which were normalized with respect to the mean ADC in healthy tissue (ADCₕ) reported in each of them. The model equation is shown in the lower left inlet. A continuous, extended GS scale is leveraged, in which 0<GS<2 indicates healthy tissue and GS>2 corresponds to PCa (i.e., overlapping the standard discrete values of the GS used to assess histopathological samples of PCa). FIG. 12B shows the mapping of the hyperbolic tangent model obtained for the normalized ADC values (ADC/ADCₕ, panel A) to normalized tumor cell density values (i.e., N/θ). A linear mapping is used that introduces a negative proportionality between ADC and N, as observed in the literature and as proposed for other biomechanistic models of solid tumor growth. The equation for this mapping is show in the lower right inlet. ADCₘᵢₙ is the value of the lower horizontal asymptote of the hyperbolic tangent model shown in panel A.

FIG. 13 depicts a biomechanistic model of PCa growth during AS, according to some embodiments. In the illustrated embodiment, the growth of newly-diagnosed untreated prostatic tumors is described in terms of the dynamics of tumor cell density (N), which may vary between N=0 far from the tumor to a maximum value of N=θ inside the tumor. The dynamics of PCa cell density is governed by the two mechanisms shown in this figure: (i) tumor cell mobility, which aims at expanding the size of the tumor and is represented with a diffusion process controlled by parameter D (i.e., the tumor cell mobility coefficient); and (ii) tumor cell net proliferation, which increases the local tumor cell density and is modeled as a logistic growth process controlled by parameter ρ (i.e., tumor cell net proliferation rate). Since newly-diagnosed PCa cases are organ-confined, it may further be assumed that tumor cell cannot leave the patient's prostate by enforcing ∇N·n=0.

The following numbered clauses set out various non-limiting embodiments disclosed herein:

Set A

A1. A method of modelling implementing one or more of the reaction-diffusion equations as described herein, wherein the reaction-diffusion equations include: an equation representing dynamics of the tumor cell density field N(x,t). an equation representing dynamics of a nutrient field s, and/or an equation representing tissue PSA p, wherein a value N(x,t) of the tumor cell density field N represents the number of tumor cells per unit volume of tissue at position x and time t, wherein a value s(x,t) of the nutrient field s represents a nutrient concentration at position x and time t, and wherein a value p(x,t) of the tissue PSA p represents the value of this variable at position x and time t.

A2. The method according to any previous clause within set A, wherein there is a reaction-diffusion equation describing the dynamics of the tumor cell density field for each of the tumors within the patient's prostate.

A3. The method according to any previous clause within set A, wherein the tumor cell density fields are initialized from ADC maps obtained from DW-MRI.

A4. The method according to any previous clause within set A, wherein the tissue PSA field is initialized from the serum PSA values collected for the individual patient.

A5. The method according to any previous clause within set A, wherein the operations include the sampling of parameter values in the equations of the model to perform model simulations for each individual patient and, hence, obtain a collection of forecasts of the spatiotemporal tumor cell density field, nutrient field, and tissue PSA field in the future.

A6. The method according to any previous clause within set A, wherein operations include any of the following calculations in any timepoint of the forecasts:
a. The spatial integration of the region of tissue where the tumor cell density field is larger than a user-specified threshold to calculate the tumor volume;
b. The spatial integration of the tumor cell density field to obtain the total tumor cell count;
c. The spatial integration of the tumor cell density field normalized to the tissue carrying capacity to obtain the total tumor cell volume;
d. The calculation of the (normalized) tumor cell density over the region of prostate tissue where the tumor cell density is larger than a user-specified threshold;
e. The calculation of the total tumor index as the product of the normalized tumor cell density times the tumor volume and divided by the prostate volume;
f. The calculation of the mean proliferation activity of the tumor by spatially integrating the terms in the tumor dynamics equation representing total or net proliferation;
g. The spatial integration of the tissue PSA field to obtain serum PSA values;
h. The spatial integration of the time derivative of the tissue PSA to obtain PSA velocity values.

A7. The method according to any previous clause within set A, wherein operations include any of the following calculations in any timepoint of the forecasts:
a. The statistical analysis of the forecasts (e.g., the fields from A5 and/or the quantities in A6) to obtain probabilities of the tumor reaching specific parts of the prostate, invading an area of the prostate external boundary, and other features including, but not limited to:
   penetrate a capsule of the prostate gland of the subject;
   invade a tissue outside (or near) the prostate gland of the subject;
   reach a specified part of the prostate gland of the subject;
   transition from ellipsoidal growth to fingered growth; or
   make contact with the urethra of the subject; and
   outputting the real-world time or an indication of the real-world time via an output device;
b. The statistical analysis of the forecasts to obtain probabilities of tumor progression to high-risk disease.

A8. The method according to any previous clause within set A, wherein operations include any of the following:
a. The prescription of PSA tests, mpMRI scans, and/or biopsies based on the forecasts (i.e., the fields from A5 and/or the quantities in A6) and/or the statistical analyses in A7.
b. The design of a personalized monitoring plan based on the forecasts (i.e., the fields from claim V and/or the quantities in A6) and/or the statistical analyses in A7).
c. The prescription of treatment based on the forecasts (i.e., the fields from A5 and/or the quantities in A6) and/or the statistical analyses in A7.
d. The design of an optimal treatment plan based on the forecasts (i.e., the fields from A5 and/or the quantities in A6) and/or the statistical analyses A7.

A9. The method according to any previous clause within set A, wherein the parameters in the equations governing the dynamics of tumor cell density are calculated in a personalized manner by using a calibration method informed by longitudinal T2 W-MRI and DW-MRI data collected for each individual patient.

A10. The method according to any previous clause within set A, wherein the parameters in the equation governing the dynamics of tissue PSA are calculated in a personalized manner by using a calibration method informed by longitudinal serum PSA measurements collected for each individual patient.

A11. The method according to any previous clause within set A, wherein operations include the simulation of the patient-specific calibrated model to obtain a prediction of the tumor cell density field, nutrient field, and tissue PSA field in the future.

A12. The method according to any previous clause within set A, wherein operations include the following calculations in any timepoint of the forecasts obtained with a patient-specific calibrated model:
a. The spatial integration of the region of tissue where the tumor cell density field is larger than a user-specified threshold to calculate the tumor volume;
b. The spatial integration of the tumor cell density field to obtain the total tumor cell count;
c. The spatial integration of the tumor cell density field normalized to the tissue carrying capacity to obtain the total tumor cell volume;
d. The calculation of the (normalized) tumor cell density over the region of prostate tissue where the tumor cell density is larger than a user-specified threshold;
e. The calculation of the total tumor index as the product of the normalized tumor cell density times the tumor volume and divided by the prostate volume;
f. The calculation of the mean proliferation activity of the tumor by spatially integrating the terms in the tumor dynamics equation representing total or net proliferation;
g. The spatial integration of the tissue PSA field to obtain serum PSA values;
h. The spatial integration of the time derivative of the tissue PSA to obtain PSA velocity values.

A13. The method according to any previous clause within set A, wherein operations include any of the following calculations in any timepoint of the forecasts:
a. The statistical analysis of the forecasts (i.e., the fields from A11 and/or the quantities in A12) to obtain an estimation of reaching specific parts of the prostate, invading an area of the prostate external boundary, other features including, but not limited to:
   penetrate a capsule of the prostate gland of the subject;
   invade a tissue outside (or near) the prostate gland of the subject;
   reach a specified part of the prostate gland of the subject;
   transition from ellipsoidal growth to fingered growth; or
   make contact with the urethra of the subject; and
   outputting the real-world time or an indication of the real-world time via an output device;
b. The statistical analysis of the forecasts to obtain probabilities of tumor progression to high-risk disease.

A14. The method according to any previous clause within set A, wherein operations with a patient-specific calibrated model include any of the following:
a. The prescription of PSA tests, mpMRI scans, and/or biopsies based on the forecasts (i.e., the fields from A11 and/or the quantities in A12) and/or the statistical analyses in A13;
b. The design of a personalized monitoring plan based on the forecasts (i.e., the fields from A11 and/or the quantities in A12) and/or the statistical analyses in A13;
c. The prescription of treatment based on the forecasts (i.e., the fields from A11 and/or the quantities in A12) and/or the statistical analyses A13;
d. The design of an optimal treatment plan based on the forecasts (i.e., the fields from A11 and/or the quantities in A12) and/or the statistical analyses in A13.

A15. The method according to any previous clause within set A, wherein the calibration and forecasting methods include the estimation of the uncertainty of the tumor cell density field, nutrient field, tissue PSA field, and any physical or statistical quantities calculated from them.

A16. The method according to any previous clause within set A, where the uncertainties enable the calculation of confidence intervals, probabilities, and statistical risks to detect tumor progression or relevant clinical events, such as when the tumor may:
penetrate a capsule of the prostate gland of the subject;
invade a tissue outside (or near) the prostate gland of the subject;
reach a specified part of the prostate gland of the subject;
transition from ellipsoidal growth to fingered growth; or
make contact with the urethra of the subject; and
outputting the real-world time or an indication of the real-world time via an output device.

A17. The method according to any previous clause within set A, wherein operations include the use of uncertainty, confidence intervals, or statistical risks to decide for each individual patient:
a. The risk of prostate cancer progression;
b. The prescription of PSA tests, mpMRI scans, and/or biopsies;
c. The design of a personalized monitoring plan;
d. The prescription of treatment;
e. The design of an optimal treatment.

A18. The method according to any previous clause within set A, wherein the tumor cell density and the ADC of the patient can be mapped to Gleason score values using a mathematical function based on a hyperbolic tangent, thereby obtaining Gleason score maps over the tumor/tumors in the patient prostate.

A19. The method according to any previous clause within set A, wherein operations including guiding a biopsy needle to obtain tissue samples from the regions of the prostate estimated to harbor healthy tissue, the maximum estimated Gleason score, or any user-specified Gleason score.

A20. The method according to any previous clause within set A, any of the methods above, in which the model is extended to include the mechanical inhibition of tumor cell density dynamics and to include the calculation of the mechanical displacements and stresses in the prostate as described herein (e.g., as set forth in Set B below).

Set B

B1. A method using the tumor dynamics model described herein and extended with features described herein to account for the mechanical deformation of the prostate due to coexisting prostate cancer and benign prostatic hyperplasia, as well as the effect of the resulting mechanical stresses on tumor dynamics. The method may further implement any features from any previous clause within set A.

B2. The method according to any previous clause within set B, further extended to accommodate the effects of a 5-alpha reductase inhibitor to evaluate whether this treatment may result in a patient's radiologically and histopathologically-confirmed tumor exhibiting faster dynamics indicative of increased malignancy.

B3. The method according to any previous clause within set B, further extended to accommodate the effects of a 5-alpha reductase inhibitor to evaluate whether this treatment may result in a patient's radiologically detected prostatic lesion suspicious of being a tumor exhibiting faster dynamics indicative of increased malignancy.

B4. The method according to any previous clause within set B, further extended to accommodate the effects of a 5-alpha reductase inhibitor to design an optimal therapeutic plan for this drug that maximizes prostate shrinkage while minimizing the development of the patient's prostatic tumor or radiologically-detected prostatic lesion suspicious of being a tumor.

B5. The method according to any previous clause within set B, wherein the methods above are used to determine the timing of imaging scans or tumor biopsies to monitor the patient's tumor (or radiologically detected prostatic lesion suspicious of being a tumor) in the absence of as well as during and after treatments involving a 5-alpha reductase inhibitor.

B6. The method according to any previous clause within set B, wherein the methods above are used to perform longitudinal, non-rigid registration of imaging data of the prostate of each individual patient.

B7. The method according to any previous clause within set B, wherein the methods above are used to assess the progression of benign prostatic hyperplasia.

B8. The method according to any previous clause within set B, wherein the methods above are used to assess the progression of prostate cancer during active surveillance and plan treatments for prostate cancer (e.g., surgery or the definition of a radiation plan for radiotherapy).

B9. The method according to any previous clause within set B, wherein the methods above are used to detect an abnormal deformation of the prostate indicative of a growing prostatic tumor and guide the performance of ensuing biopsy to confirm this pathology.

Embodiments of the present disclosure may be realized in any of various forms. For example, some embodiments may be realized as a computer-implemented method, as a computer-readable memory medium, or as a computer system. Other embodiments may be realized using one or more custom-designed hardware devices such as ASICs. Still other embodiments may be realized using one or more programmable hardware elements such as FPGAs.

In some embodiments, a non-transitory computer-readable memory medium may be configured so that it stores program instructions and/or data, where the program instructions, when executed by a computer system, cause the computer system to perform a method, e.g., any of a method embodiments described herein, or, any combination of the method embodiments described herein, or, any subset of any of the method embodiments described herein, or, any combination of such subsets.

In some embodiments, a computer system may be configured to include a processor (or a set of processors) and a memory medium, where the memory medium stores program instructions, where the processor is configured to read and execute the program instructions from the memory medium, where the program instructions are executable to implement a method, e.g., any of the various method embodiments described herein (or, any combination of the method embodiments described herein, or, any subset of any of the method embodiments described herein, or, any combination of such subsets).

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of modeling the possible progression of prostate cancer in a subject comprising:
performing operations on a computer system to generate a tumor growth model for a prostate gland of the subject, wherein the operations include:
simulating evolution of a tumor in the prostate gland of the subject, wherein said simulating of the evolution of the tumor is based at least on a coupled system of reaction-diffusion equations and a patient-specific geometric model of the prostate gland of the subject, wherein at least one of the reaction-diffusion equations is an equation representing dynamics of tumor cell density field N(x,t) or an equation representing dynamics of a nutrient field s, and wherein a value N(x,t) of the tumor cell density field N represents a number of tumor cells per unit volume of tissue at position x and time t, and wherein the value s(x,t) of the nutrient field represents a nutrient concentration at position x and time t.

2. The method of claim 1, wherein the reaction-diffusion equations include at least one equation representing the dynamics of the tumor cell density field for each tumor present in the prostate gland.

3. The method of claim 1, wherein the tumor cell density field is initialized from apparent diffusion coefficient (ADC) maps obtained by diffusion-weighted magnetic resonance imaging (DW-MRI), and wherein the patient-specific parameters governing the equation describing the dynamics of the tumor cell density field are obtained by solving an inverse problem using one or more ADC maps.

4. The method of claim 1, wherein the at least one of the reaction-diffusion equations includes an equation representing dynamics of tissue prostate-specific antigen (PSA), a value p(x,t) of the tissue PSA field p representing a tissue PSA concentration at position x and time t, wherein the tissue PSA field is initialized from serum PSA values collected for the subject, and wherein the patient-specific parameters governing the equation describing the dynamics of tissue PSA are determined by solving an inverse problem using one or more serum PSA values.

5. The method of claim 1, wherein the tumor growth model is implemented for one or more of the following:
determining an existence, risk, and/or probability of prostate cancer progression;
determining an existence, risk, and/or probability of extracapsular extension;
determining a personalized monitoring plan for assessing growth of the tumor, wherein the monitoring plan includes timing of imaging scans of the tumor and/or tumor biopsies and/or PSA tests;
determining the Gleason score for each tumor in the prostate;
determining a spatial map of local Gleason score values for each tumor region of interest within the prostate gland;
planning biopsies of the prostate gland;
determining a decision to prescribe a treatment; and
designing an optimal treatment plans for treating the subject, wherein optimal treatment plan includes a type of treatment and its clinical implementation.

6. A method of modeling the possible progression of prostate cancer in a subject comprising:
performing operations on a computer system to generate a tumor growth model for a prostate gland of the subject, wherein the operations include:
simulating evolution of a tumor in the prostate gland of the subject, wherein said simulating of the evolution of the tumor is based at least on a coupled system of reaction-diffusion equations and a patient-specific geometric model of the prostate gland of the subject, wherein the reaction-diffusion equations include:
at least one equation representing the dynamics of the tumor;
at least one equation representing dynamics of tissue prostate-specific antigen (PSA);
at least one equation of mechanical equilibrium in the prostate gland; and
at least one equation for a mechanotransductive function that adjusts mobility and net proliferation in the evolution of the tumor.

7. The method of claim 6, wherein the at least one equation representing the dynamics of the tumor is one of:
an equation representing dynamics of an evolution of a tumor phase field ϕ, wherein a value ϕ(x,t) of the tumor phase field ϕ represents an extent to which cells at position x and time t are cancerous; and
an equation representing dynamics of tumor cell density field N(x,t), wherein a value N(x,t) of the tumor cell density field N represents a number of tumor cells per unit volume of tissue at position x and time t.

8. The method of claim 6, wherein the at least one equation of mechanical equilibrium is implemented to account for the mechanical deformation of the prostate gland during the evolution of the tumor.

9. The method of claim 8, wherein the mechanical deformation of the prostate gland is due to coexisting prostate cancer and benign prostatic hyperplasia.

10. The method of claim 6, wherein the at least one equation for the mechanotransductive function is implemented to account for mechanical stresses on the evolution of the tumor.

11. The method of claim 10, wherein the at least one equation for the mechanotransductive function implements at least one scalar metric that summarizes mechanical stress across the prostate gland.

12. The method of claim 6, wherein the at least one equation of mechanical equilibrium includes a term that represents stresses induced by the evolution of the tumor, wherein the term is a function of a variable representing position of tumor tissue.

13. The method of claim 6, wherein the at least one equation of mechanical equilibrium includes a term that represents stresses induced by benign prostatic hyperplasia, wherein the term is defined over a central gland of the prostate and is a function of a growth rate of the central gland over time.

14. The method of claim 6, wherein simulating evolution of the tumor in the prostate gland of the subject includes assessing effects of a 5-alpha reductase inhibitor on the evolution of the tumor.

15. The method of claim 14, wherein assessing effects of the 5-alpha reductase inhibitor includes determining whether the tumor or the serum PSA exhibit faster dynamics.

16. The method of claim 14, wherein assessing effects of the 5-alpha reductase inhibitor includes implementing a factor representing a drug-induced increase in tumor cell death in the at least one equation representing tumor dynamics.

17. The method of claim 14, wherein assessing effects of the 5-alpha reductase inhibitor includes implementing a term representing inhibition of benign prostatic hyperplasia and drug-induced shrinkage of the prostate gland.

18. The method of claim 6, further comprising developing a therapeutic plan for treatment of the subject using a 5-alpha reductase inhibitor based on the tumor growth model.

19. The method of claim 6, further comprising implementing use of personalized model simulations for one or more of the following:
determining a personalized monitoring plan for assessing growth of the tumor, wherein the monitoring plan includes timing of imaging scans of the tumor and/or tumor biopsies and/or serum PSA tests;
planning the treatment of prostate cancer;
planning the treatment of benign prostatic hyperplasia; and
planning biopsies of the prostate gland.

20. The method of claim 6, further comprising performing longitudinal, non-rigid registration of imaging data of the prostate of the subject based on the tumor growth model, wherein personalized model simulations and the longitudinal, non-rigid registration of imaging data are implemented in one or more of the following:
assessing progression of benign prostatic hyperplasia in the prostate gland;
assessing progression of prostate cancer in the prostate gland;
determining a personalized monitoring plan for assessing growth of the tumor, wherein the monitoring plan includes timing of imaging scans of the tumor, tumor biopsies, and/or serum PSA tests;
planning the treatment of prostate cancer;
planning the treatment of benign prostatic hyperplasia;
detecting undiagnosed prostate cancer causing an abnormal deformation of the prostate gland; and
planning biopsies of the prostate gland.
